(19) United States
(12) Patent Application Publication
(10) Pub. No.: US 2008/0269285 A1
(43) Pub. Date: Oct. 30, 2008

(54) Novel Compounds of the Family of Iminosugars, Uses Thereof for Trewating Lysosomal Diseases, and Method for Preparing Same

(75) Inventors: Olivier Richard Martin, Hilaire Saint Mesmin (FR); Philippe Compain, Orleans (FR); Charlotte Boucheron, Orleans (FR); Naoki Asano, Kanazawa (JP)

Correspondence Address: YOUNG & THOMPSON, 209 Madison Street, Suite 500, ALEXANDRIA, VA, 22314, US

(73) Assignee: CENTRE NATIONAL DE LA RECHERCHE SCIENTIFIQUE, Paris (FR)

(21) Appl. No.: 11/993,880

(22) PCT Filed: Jun. 22, 2006
(86) PCT No.: PCT/FR06/01425

§ 371 Date: Mar. 6, 2008

(30) Foreign Application Priority Data
Jun. 23, 2005 (FR) 0506382

(51) Int. Cl. A61K 31/45; C07D 211/46; A61P 43/00

(52) U.S. Cl. 514/315; 546/242

(57) ABSTRACT

The invention concerns the use of a compound of general formula (I) wherein: R₀ represents in particular a hydrogen atom or an alkyl group; R₁ represents in particular a hydrogen atom or an alkyl group; R₂, R₃ and R₄ represent in particular, independently of one another, a hydrogen atom, a hydroxyl group, an alkoxy group or an acyloxy group, for preparing a medicine for treating lysosomal diseases related to a dysfunction of at least one lysosomal glycosidase enzyme.

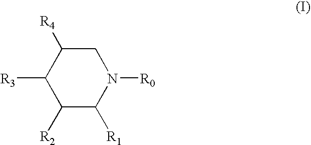

A subject of the present invention is novel compounds of the iminosugar family, as well as their preparation process.

A subject of the present invention is also the use of these novel compounds of the iminosugar family, in particular within the framework of the treatment of lysosomal diseases.

Lysosomal diseases are hereditary diseases which are characterized by deficiency in an enzyme involved in the catabolism of glycosphingolipids within lysosomes; this degradation process is due to the action of a series of glycosidases which hydrolyse the glycoside bonds present in the glycosphingolipids in order to finally lead to ceramide release. The dysfunction of one or other of these glycosidases is the cause of lysosomal diseases such as for example Gaucher's disease.

Gaucher's disease is a rare hereditary disease which affects one person in 50,000 world-wide but which occurs much more frequently in the Ashkenazi Jewish community with almost one person in 500 (Futerman, A. H.; Sussman, J. L.; Horowitz, M.; Silman, I.; Zimran, A. Novel directions in the treatment of Gaucher's disease Trends in Pharm. Sci. 2004, 25, 147). Gaucher's disease originates in the alteration of the catalytic activity of β-glucocerebrosidase and leads to the accumulation of glucosylceramide in different tissues and, progressively, to severe dysfunctions in particular of a neuropathological or psychomotor nature which can result in death before adulthood in certain cases.

The most used therapeutic strategy involves injecting a recombinant enzyme, Ceredase®, by intravenous route in order to compensate for the activity of the deficient enzyme (ERT: “enzyme replacement therapy”: Grabowski, G. A; Hopkin, R. J. Enzyme therapy for lysosomal storage disease: principle, practice and prospect. Annu. Rev. Genomics Human Genet. 2003, 4, 403). The cost of this therapy is extremely high. Moreover, the latter does not make it possible to treat the neurological forms of the disease and has little effect on patients whose bones and lungs are affected (Grabowski, G. A; Hopkin, R. J. Enzyme therapy for lysosomal storage disease: principle, practice and prospect. Annu. Rev. Genomics Human Genet. 2003, 4, 403).

The second strategy uses an iminosugar, N-butyl-1-deoxynojirimycin, as active ingredient of a medicament, Zavesca®. This compound acts by limiting the biosynthesis of the glycosphingolipids and thus reducing the quantity of glucosylceramide, the natural glucocerebrosidase substrate (SRT: “substrate reduction therapy”: Cox et al., Novel oral treatment of Gaucher's disease with N-butyldeoxynojirimycin (OGT 918) to decrease substrate biosynthesis. Lancet 2000, 355, 1481). This oral treatment leads to numerous side-effects and is indicated only in the case where Ceredase® cannot be used. Thus, Zavesca® is contra-indicated for certain categories of persons (children, adolescents and pregnant women) and the treatment is accompanied by various drawbacks linked mainly to the inhibition of intestinal glucosidases (loss of weight, abdominal pain, diarrhoea). This medicament blocks spermatogenesis (Van der Spoel et al., Reversible infertility in male mice after oral administration of alkylated imino sugars: a nonhormonal approach to male contraception. Proc. Nat. Acad Sci. USA 2002, 99, 17173) and large doses (100-300 mg) must be used every day (Zimran, A.; Elstein, D. Gaucher's disease and the clinical experience with substrate reduction therapy. Philos. Trans. R. Soc. Lond. B Biol. Sci. 2003, 355, 961).

Thus, the purpose of the present invention is to provide novel compounds of iminosugar type, having a very strong inhibition activity on the enzyme β-glucocerebrosidase.

Another purpose of the invention involves providing novel compounds which are useful within the framework of the treatment of lysosomal diseases, in particular of Gaucher's disease, said compounds having a very strong inhibition activity on the enzyme β-glucocerebrosidase, at lower doses than within the framework of the use of the products currently used for the treatment of such diseases.

The present invention relates to the use of a compound of the following general formula (I):

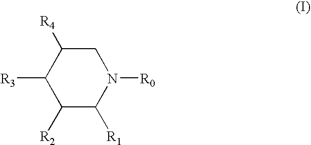

in which:
R₀ represents:
   a hydrogen atom, or
   a linear or branched, saturated or unsaturated alkyl group, comprising 1 to 12 carbon atoms, and preferably comprising 4 to 12 carbon atoms, in particular 6 to 12 carbon atoms, said alkyl group being optionally substituted by a phenyl group, if appropriate substituted by an alkoxy group comprising 1 to 15 carbon atoms, or
   an n-oxaalkyl group comprising 3 to 12 members,
R₁ represents:
   a hydrogen atom, or
   a linear or branched, saturated or unsaturated alkyl group, comprising 4 to 16 carbon atoms, said alkyl group being optionally substituted by or carrying a substituent chosen from the following groups: hydroxyl, alkoxy comprising 1 to 12 carbon atoms and phenyl, or
   an n-oxaalkyl group comprising 4 to 12 members, n representing an integer greater than or equal to 3,
R₂, R₃ and R₄ represent, independently of one another:
   a hydrogen atom, or
   a hydroxyl group, or
   an alkoxy group of formula OR₅, R₅ representing a linear or branched, saturated or unsaturated alkyl group, comprising 1 to 15 carbon atoms, preferably comprising 4 to 12 carbon atoms, or a benzyl group, or
   an acyloxy group of formula O—CO—R₆, R₆ representing a linear or branched, saturated or unsaturated alkyl group, comprising 1 to 15 carbon atoms,

at least one of the R₁, R₂, R₃ and R₄ groups representing a linear or branched, saturated or unsaturated alkyl group, comprising 4 to 16 carbon atoms as defined above, or representing a group comprising a linear or branched, saturated or unsaturated alkyl group, comprising 1 to 15 carbon atoms as defined above,

said compound of formula (I) being in the form of a pure stereoisomer or in the form of a mixture of enantiomers and/or diastereoisomers, including a racemic mixture as well as their addition salts with pharmacologically acceptable acids,

for the preparation of a medicament intended for the treatment of lysosomal diseases linked to a dysfunction of at least one lysosomal glycosidase enzyme,
providing that:
   in formula (I), when R₀ represents a hydrogen atom or an alkyl group comprising 1 to 3 carbon atoms and R₁ represents a hydrogen atom, at least one of the R₂, R₃ and R₄ groups represents an alkoxy OR₅ or acyloxy OCOR₆ group as defined above, in which R₅ or R₆ represents an alkyl group comprising at least 3 carbon atoms, and
   in formula (I), at least two of the R₂, R₃ and R₄ groups do not represent a hydrogen atom.

The expression “n-oxaalkyl” designates an alkyl chain in which the nth —CH₂— group is replaced by an oxygen atom. Among the n-oxaalkyl groups, there can be mentioned the 5-oxanonyl group, corresponding to a nonyl chain in which the fifth CH₂ group is replaced by an oxygen atom; such a group corresponds to the following formula: —CH₂—CH₂—CH₂—CH₂—O—CH₂—CH₂—CH₂—CH₃.

The expression “addition salts with pharmacologically acceptable acids” designates the salts of the compounds of general formula (I) formed by the addition of acids the anions of which form non-toxic salts, such as for example the salts formed with hydrochloric, sulphuric, phosphoric, acetic, lactic, citric, tartaric, gluconic and saccharic acids.

The expression “lysosomal diseases linked to a dysfunction of at least one lysosomal glycosidase enzyme” designates hereditary diseases which are characterized by deficiency in an enzyme involved in the catabolism of the glycosphingolipids within lysosomes; for example, Gaucher's disease is linked to the dysfunction of β-glucocerebrosidase, Fabry's disease to the dysfunction of α-galactosidase A, Krabbe's disease to the dysfunction of β-galactocerebrosidase, Tay-Sachs disease to the dysfunction of β-hexosaminidase A and Sandhoff's disease to the dysfunction of β-hexosaminidase B.

The therapeutic strategy implemented within the framework of the present invention involves using compounds which act as a “chemical chaperone” of the deficient mutant enzyme by stabilizing its three-dimensional structure (Fan, J.-Q. A contradictory treatment for lysosomal storage disorders: inhibitors enhance mutant enzyme activity, Trends Pharm. Sci, 2003, 24, 355) The use of these compounds at a very low concentration is capable of increasing the in vivo residual hydrolytic activity of the mutant enzyme and thus reducing the accumulation of the glucosylceramide involved in Gaucher's disease. This approach has numerous advantages: oral treatment, no envisaged side-effect (the compounds of the invention are extremely specific to β-glucocerebrosidase and the doses used are very low: of the order of 10⁻⁹ molar in cell tests).

The present invention relates to the use as defined above, for the preparation of a medicament intended for the treatment of Gaucher's disease.

Gaucher's disease is characterized by a deficiency in β-glucocerebrosidase, a lysosomal enzyme which catalyzes the conversion of glucocerebroside to glucose and ceramide. Glucocerebroside is a complex lipid, a constituent of cell membranes, essentially originating from degradation of the erythrocytes. The clinical manifestations of the disease are secondary to its accumulation in the tissues. The elimination of the glucocerebroside is usually carried out in the cells of the reticuloendothelial system, which adopt, during the course of the disease, a characteristic morphology (Gaucher cells) due to accumulation of glucocerebroside in the lysosomes. The hystiocytes of the spleen, the Küpfer cells of the liver, the macrophages of the bone marrow and the periadventitial cells of the Virchow-Robin spaces in the brain are involved.

The present invention relates to the use as defined above, for the preparation of a medicament intended for the treatment of Krabbe's disease.

Krabbe's disease or globoid cell leukodystrophy is a disease with recessive autosomic transmission, as a consequence of a deficiency in galactocerebrosidase, a lysosomal enzyme involved in the catabolism of a major lipid constituent of myelin. Its frequency appears to be of the order of 1/150,000 births in France. The disease leads to a demyelinization of the central and peripheral nervous system.

The present invention relates to the use as defined above, for the preparation of a medicament intended for the treatment of Fabry's disease.

Fabry's disease is a hereditary pathology of the metabolism of the glycosphingolipids, with recessive transmission linked to the X chromosome, due to an α-galactosidase A deficiency. The enzymatic defect leads to the accumulation of the non-degraded substrate in the tissues and the plasma. In its standard form, the disease more severely affects hemizygous men, in whom the clinical signs start in childhood with pain in the extremities and dermatological signs (angiokeratomas). Subsequently, a multivisceral overload disease develops with cardiac (left ventricular hypertrophy), neurological (cerebrovascular accidents), ORL (hypoacousia) and renal symptoms (proteinuria, renal insufficiency).

The present invention relates more particularly to the use as defined above of a compound of general formula (I-A), corresponding to the abovementioned formula (I) in which:
R₀ represents:
   a hydrogen atom, or
   a linear or branched, saturated or unsaturated alkyl group, comprising 1 to 12 carbon atoms, and preferably comprising 4 to 12 carbon atoms, in particular 6 to 12 carbon atoms, said alkyl group being optionally substituted by a phenyl group, if appropriate substituted by an alkoxy group comprising 1 to 15 carbon atoms,
R₁ represents:
   a hydrogen atom, or
   a linear or branched, saturated or unsaturated alkyl group, comprising 4 to 16 carbon atoms,
R₂, R₃ and R₄ represent, independently of one another:
   a hydroxyl group, or
   an alkoxy group of formula OR₅, R₅ representing a linear or branched, saturated or unsaturated alkyl group, comprising 1 to 15 carbon atoms, preferably comprising 4 to 12 carbon atoms, or a benzyl group.

According to an advantageous embodiment, the present invention relates to the use as defined above, of compounds of formula (I) or (I-A) in which R₀ represents a hydrogen atom.

The present invention therefore relates to the use as defined above of compounds corresponding to the following formula (I-1):

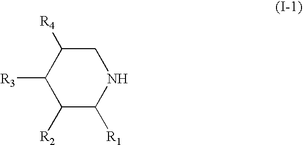

R₁, R₂, R₃ and R₄ being as defined above for the compounds of formula (I) or (I-A).

According to an advantageous embodiment, the present invention relates to the use as defined above, of compounds of formula (I-1) in which R₁ represents an alkyl group comprising 4 to 16 carbon atoms, and preferably comprising 9 carbon atoms.

According to an advantageous embodiment, the present invention relates to the use of the following compound:

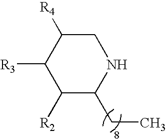

This compound corresponds to a compound of formula (I-1) as defined above, in which R₁ represents a nonyl group, R₂, R₃ and R₄ being as defined above.

According to an advantageous embodiment, the present invention relates to the use as defined above, of compounds of formula (I-1) in which R₂, R₃ and R₄ represent an OH group.

The present invention therefore relates to the use as defined above of compounds corresponding to the following formula (I-2):

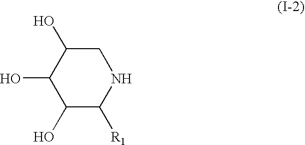

R₁ being as defined above for the compounds of formula (I) or (I-A).

According to an advantageous embodiment, the present invention relates to the use of the following compound:

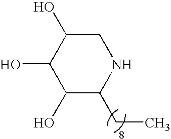

This compound corresponds to a compound of formula (I-2) as defined above, in which R₁ represents a nonyl group.

The present invention also relates to the use as defined above, of compounds of formula (I) or (I-A) in which R₀ represents an alkyl group comprising 6 to 12 carbon atoms, and preferably comprising 9 carbon atoms.

Thus, advantageously, the present invention relates to the use of a compound of the following formula:

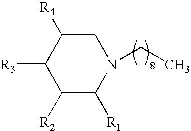

This compound corresponds to a compound of formula (I) or (I-A) as defined above, in which R₀ represents a nonyl group, R₁, R₂, R₃ and R₄ being as defined above for the compounds of formula (I) or (I-A).

The present invention also relates to the use as defined above, of compounds of formula (I) or (I-A), in which R₀ represents an alkyl group as defined above and R₁ represents a hydrogen atom.

The present invention therefore relates to the use as defined above of compounds corresponding to the following formula (I-3):

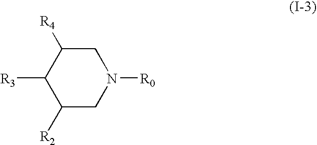

in which R₂, R₃ and R₄ are as defined above for the compounds of formula (I) or (I-A).

Advantageously, the present invention relates to the use as defined above of the following compound:

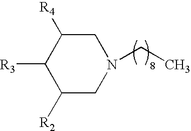

in which R₂, R₃ and R₄ are as defined above for the compounds of formula (I) or (I-A).

According to an advantageous embodiment, the present invention relates to the use as defined above, of compounds of formula (I) or (I-A) in which R₂ represents an alkoxy group of formula OR₅, R₅ representing an alkyl group comprising 3 to 15 carbon atoms, preferably comprising 4 to 12 carbon atoms.

According to an advantageous embodiment, the present invention relates to the use as defined above, of compounds of formula (I) or (I-A) in which R₀ represents an alkyl group as defined above and R₂ represents an alkoxy group of formula OR₅, R₅ representing an alkyl group comprising 3 to 15 carbon atoms, preferably comprising 4 to 12 carbon atoms.

According to another advantageous embodiment, the present invention relates to the use as defined above, of compounds of formula (I) or (I-A) in which R₀ represents an alkyl group as defined above, R₁ represents a hydrogen atom and R₂ represents an alkoxy group of formula OR₅, R₅ representing an alkyl group comprising 3 to 15 carbon atoms, preferably comprising 4 to 12 carbon atoms. Thus, the present invention relates to the use of compounds of the abovementioned formula (I-3), in which R₂ represents an alkoxy group of formula OR₅ as defined above.

According to an advantageous embodiment, the present invention relates to the use as defined above, characterized in that R₃ and R₄ represent OH groups.

The present invention therefore relates to the use as defined above of compounds corresponding to the following formula (I-4):

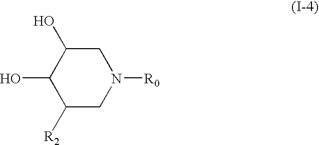

in which R₀ represents an alkyl group as defined above, and R₂ is as defined above for the compounds of formula (I) or (I-A), and preferably represents an alkoxy group, OR₅ as defined above.

The present invention also relates to the use as defined above, characterized in that R₃ represents an OH group and R₄ represents an alkoxy group of formula OR₅ R₅ representing an alkyl group comprising 3 to 15 carbon atoms, preferably comprising 4 to 12 carbon atoms.

The present invention therefore relates to the use as defined above of compounds corresponding to the following formula (I-5):

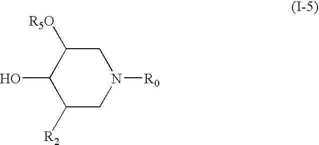

in which R₀ and R₅ represent an alkyl group as defined above, and R₂ is as defined above for the compounds of formula (I) or (I-A), and preferably represents an alkoxy group OR₅ as defined above.

The present invention also relates to the use as defined above of a compound of the following general formula:

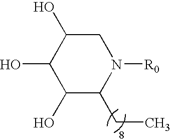

R₀ being as defined above, and representing in particular an alkyl group comprising 1 to 12 carbon atoms, preferably a butyl group, or an alkyl group as defined above, in particular a methyl group, substituted by a phenyl group, if appropriate substituted by an alkoxy group comprising 1 to 15 carbon atoms, preferably a methoxy group.

The present invention also relates to the use as defined above of a compound of the following general formula (II):

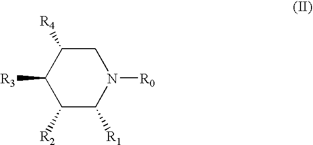

R₀, R₁, R₂, R₃ and R₄ being as defined above for the compounds of formula (I) or (I-A).

Such a compound is a derivative of 1,5-dideoxy-1,5-imino-D-xylitol.

The present invention also relates to the use as defined above of a compound of the following formula (III):

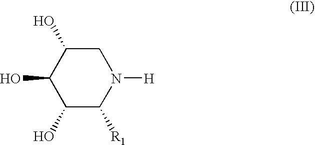

in which R₁ represents an alkyl group as defined above, and preferably a nonyl group.

The present invention also relates to the use as defined above of a compound of the following formula (IV-1):

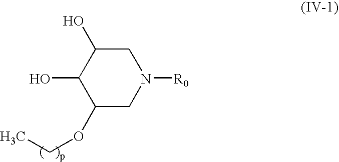

in which:
p represents an integer varying from 0 to 11, and preferably equal to 8,
R₀ represents an alkyl group as defined above, and preferably a nonyl group.

The present invention also relates to the use as defined above of a compound of the following formula (IV):

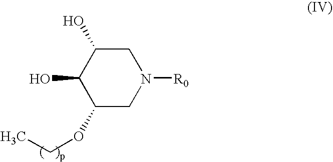

in which:
p represents an integer varying from 0 to 11, and preferably equal to 8,
R₀ represents an alkyl group as defined above, and preferably a nonyl group.

The present invention also relates to the use as defined above of a compound of the following formula (V-1):

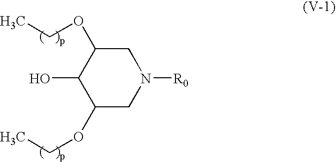

in which:
p represents an integer varying from 0 to 11, and preferably equal to 8,
R₀ represents an alkyl group as defined above, and preferably a nonyl group.

The present invention also relates to the use as defined above of a compound of the following formula (V):

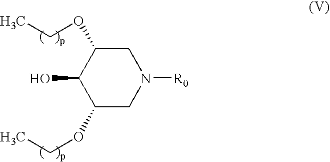

in which:
p represents an integer varying from 0 to 11, and preferably equal to 8,
R₀ represents an alkyl group as defined above, and preferably a nonyl group.

The present invention also relates to the use as defined above of a compound of the following formula (II-1):

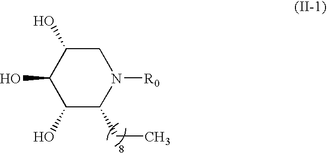

R₀ being as defined above, and representing in particular an alkyl group comprising 1 to 12 carbon atoms, preferably a butyl group, or an alkyl group as defined above, in particular a methyl group, substituted by a phenyl group, if appropriate substituted by an alkoxy group comprising 1 to 15 carbon atoms, preferably a methoxy group.

The present invention also relates to the use as defined above of a compound of the following formula (II-2):

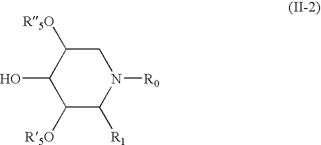

in which:
R₀ is as defined above for formula (I), and represents in particular H or an alkyl group comprising 1 to 12 carbon atoms, preferably a butyl, octyl or nonyl group, or an alkyl group, in particular a methyl group, substituted by a phenyl group, if appropriate substituted by an alkoxy group comprising 1 to 15 carbon atoms, preferably a methoxy group,
R₁ is as defined above for formula (I), and represents in particular H or an alkyl group comprising 4 to 16 carbon atoms, preferably a nonyl group,
R′₅ and R″₅ represent independently of one another H or an alkyl group comprising 1 to 15 carbon atoms, preferably an octyl or nonyl group.

The present invention also relates to the use as defined above of a compound of the following formula (II-3):

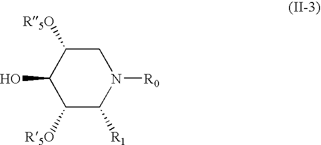

in which R₀, R₁, R′₅ and R″₅ are as defined above in formula (II-2).

The present invention also relates to a compound of the following general formula (I):

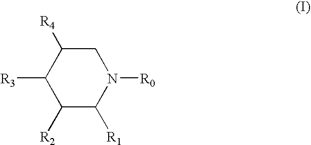

in which:
R₀ represents:
   a hydrogen atom, or
   a linear or branched, saturated or unsaturated alkyl group, comprising 1 to 12 carbon atoms, and preferably comprising 4 to 12 carbon atoms, in particular 6 to 12 carbon atoms, said alkyl group being optionally substituted by a phenyl group, if appropriate substituted by an alkoxy group comprising 1 to 15 carbon atoms, or
   an oxaalkyl group comprising 3 to 12 members,
R₁ represents:
   a hydrogen atom, or
   a linear or branched, saturated or unsaturated alkyl group, comprising 4 to 16 carbon atoms, said alkyl group being optionally substituted by or carrying a substituent chosen from the following groups: hydroxyl, alkoxy comprising 1 to 12 carbon atoms and phenyl, or
   an n-oxaalkyl group comprising 4 to 12 members, n representing an integer greater than or equal to 3,
R₂, R₃ and R₄ represent, independently of one another:
   a hydrogen atom, or
   a hydroxyl group, or
   an alkoxy group of formula OR₅, R₅ representing a linear or branched, saturated or unsaturated alkyl group, comprising 1 to 15 carbon atoms, preferably comprising 4 to 12 carbon atoms, or a benzyl group, or
   an acyloxy group of formula O—CO—R₆, R₆ representing a linear or branched, saturated or unsaturated alkyl group, comprising 1 to 15 carbon atoms,

at least one of the R₁, R₂, R₃ and R₄ groups representing a linear or branched, saturated or unsaturated alkyl group, comprising 4 to 16 carbon atoms as defined above, or representing a group comprising a linear or branched, saturated or unsaturated alkyl group, comprising 1 to 15 carbon atoms as defined above,

said compound of formula (I) being in the form of a pure stereoisomer or in the form of a mixture of enantiomers and/or of diastereoisomers, including a racemic mixture as well as their addition salts with pharmacologically acceptable acids,
providing that:
   in formula (I), when R₀ represents a hydrogen atom or an alkyl group comprising 1 to 3 carbon atoms and R₁ represents a hydrogen atom, at least one of the R₂, R₃ and R₄ groups represents an alkoxy group OR₅, or acyloxy group OCOR₆, as defined above, in which R₅ or R₆ represents an alkyl group comprising at least 3 carbon atoms, and
   in formula (I), at least two of the R₂, R₃ and R₄ groups do not represent a hydrogen atom.

The present invention relates more particularly to a compound of general formula (I-A), corresponding to the abovementioned formula (I) in which:
R₀ represents:
   a hydrogen atom, or
   a linear or branched, saturated or unsaturated alkyl group, comprising 1 to 12 carbon atoms, and preferably comprising 4 to 12 carbon atoms, in particular 6 to 12 carbon atoms, said alkyl group being optionally substituted by a phenyl group, if appropriate substituted by an alkoxy group comprising 1 to 15 carbon atoms,
R₁ represents:
   a hydrogen atom, or
   a linear or branched, saturated or unsaturated alkyl group, comprising 4 to 16 carbon atoms,
R₂, R₃ and R₄ represent, independently of one another:
   a hydroxyl group, or
   an alkoxy group of formula OR₅, R₅ representing a linear or branched, saturated or unsaturated alkyl group, comprising 1 to 15 carbon atoms, preferably comprising 4 to 12 carbon atoms, or a benzyl group.

A class of preferred compounds of the invention is constituted by compounds of formula (I) or (I-A) in which R₀ represents a hydrogen atom.

The present invention therefore relates to compounds of the following formula (I-1):

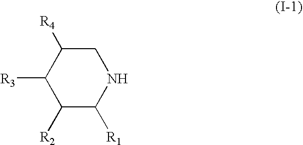

R₁, R₂, R₃ and R₄ being as defined above for the compounds of formula (I) or (I-A).

According to an advantageous embodiment, the present invention relates to compounds of formula (I-1) in which R₁ represents an alkyl group comprising 4 to 16 carbon atoms, and preferably comprising 9 carbon atoms.

Among the compounds of formula (I-1), a particularly advantageous compound is the following compound:

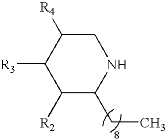

This compound corresponds to a compound of formula (I-1) as defined above, in which R₁ represents a nonyl group, R₂, R₃ and R₄ being as defined above.

According to another advantageous embodiment, the present invention relates to compounds of formula (I-1) in which R₂, R₃ and R₄ represent an OH group.

The present invention therefore relates to compounds corresponding to the following formula (I-2):

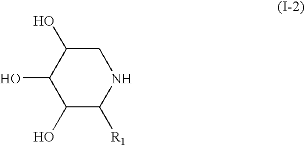

R₁ being an alkyl group as defined above for the compounds of formula (I) or (I-A).

According to an advantageous embodiment, the present invention relates to the use of the following compound:

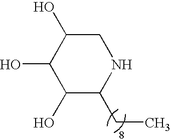

This compound corresponds to a compound of formula (I-2) as defined above, in which R₁ represents a nonyl group.

The present invention also relates to compounds of formula (I) or (I-A), in which R₀ represents an alkyl group comprising 6 to 12 carbon atoms, and preferably comprising 9 carbon atoms.

Thus, advantageously, the present invention relates to a compound of the following formula:

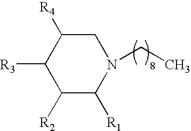

R₁, R₂, R₃ and R₄ being as defined above for the compounds of formula (I) or (I-A).

The present invention also relates to compounds of formula (I) or (I-A), in which R₀ represents an alkyl group and R₁ represents a hydrogen atom.

The present invention therefore relates to the use of the compounds corresponding to the following formula (I-3):

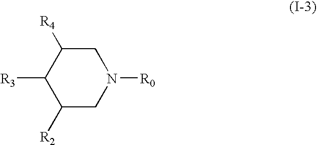

in which R₂, R₃ and R₄ are as defined above for the compounds of formula (I) or (I-A).

Advantageously, the present invention relates to the following family of compounds:

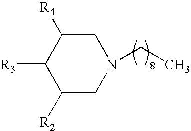

in which R₂, R₃ and R₄ are as defined above for the compounds of formula (I) or (I-A).

This compound corresponds to a compound of formula (I-3) as defined above, in which R₀ represents a nonyl group.

According to an advantageous embodiment, the present invention relates to compounds of formula (I) or (I-A) in which R₂ represents an alkoxy group of formula OR₅, R₅ representing an alkyl group comprising 3 to 15 carbon atoms, preferably comprising 4 to 12 carbon atoms.

According to another advantageous embodiment, the present invention relates to compounds of formula (I) or (I-A) in which R₀ represents an alkyl group as defined above and R₂ represents an alkoxy group of formula OR₅, R₅ representing an alkyl group comprising 3 to 15 carbon atoms, preferably comprising 4 to 12 carbon atoms.

According to another advantageous embodiment, the present invention relates to compounds of formula (I) or (I-A) in which R₀ represents an alkyl group as defined above, R₁ represents a hydrogen atom and R₂ represents an alkoxy group of formula OR₅, R₅ representing an alkyl group comprising 3 to 15 carbon atoms, preferably comprising 4 to 12 carbon atoms. Thus, the present invention relates to compounds of the abovementioned formula (I-3), in which R₂ represents an alkoxy group of formula OR₅ as defined above.

The present invention also relates to compounds of formula (I) or (I-A), in which R₃ and R₄ represent OH groups.

The present invention therefore relates to compounds corresponding to the following formula (I-4):

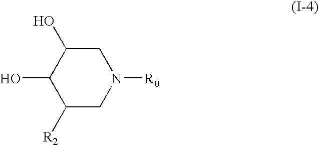

in which R₀ represents an alkyl group as defined above, and R₂ is as defined above for the compounds of formula (I) or (I-A), and preferably represents an alkoxy group OR₅ as defined above.

The present invention also relates to compounds of formula (I) or (I-A), in which R₃ represents an OH group and R₄ represents an alkoxy group of formula OR₅, R₅ representing an alkyl group comprising 3 to 15 carbon atoms, preferably comprising 4 to 12 carbon atoms.

The present invention therefore relates to compounds corresponding to the following formula (I-5):

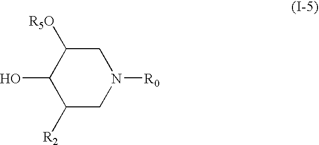

in which R₀ and R₅ represent an alkyl group as defined above, and R₂ is as defined above for the compounds of formula (I) or (I-A), and preferably represents an alkoxy group OR₅, as defined above.

The present invention also relates to a compound as defined above, corresponding to the following general formula:

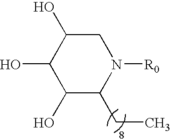

R₀ being as defined above, and representing in particular an alkyl group comprising 1 to 12 carbon atoms, preferably a butyl group, or an alkyl group as defined above, in particular a methyl group, substituted by a phenyl group, if appropriate substituted by an alkoxy group comprising 1 to 15 carbon atoms, preferably a methoxy group.

The present invention relates to a compound as defined above, corresponding to the following formula (II):

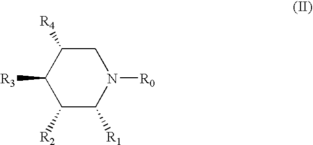

R₀, R₁, R₂, R₃ and R₄ being as defined above in formula (I) or (I-A).

The present invention also relates to a compound as defined above, corresponding to the following formula (II-1):

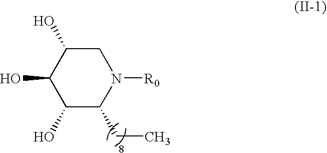

R₀ being as defined above, and representing in particular an alkyl group comprising 1 to 12 carbon atoms, preferably a butyl group, or an alkyl group as defined above, in particular a methyl group, substituted by a phenyl group, if appropriate substituted by an alkoxy group comprising 1 to 15 carbon atoms, preferably a methoxy group.

The present invention also relates to a compound as defined above, corresponding to the following formula (II-2):

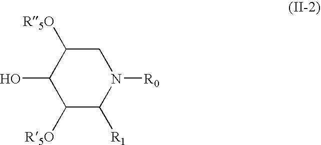

in which:
R₀ is as defined above for formula (I), and represents in particular H or an alkyl group comprising 1 to 12 carbon atoms, preferably a butyl, octyl or nonyl group, or an alkyl group, in particular a methyl group, substituted by a phenyl group, if appropriate substituted by an alkoxy group comprising 1 to 15 carbon atoms, preferably a methoxy group,
R₁ is as defined above for formula (I), and represents in particular H or an alkyl group comprising 4 to 16 carbon atoms, preferably a nonyl group,
R′₅ and R″₅ represent independently of one another H or an alkyl group comprising 1 to 15 carbon atoms, preferably an octyl or nonyl group.

The present invention relates to a compound as defined above, corresponding to the following formula (II-3):

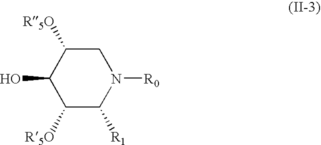

in which R₀, R₁, R′₅ and R″₅ are as defined above in formula (II-2).

The present invention also relates to a compound corresponding to the following formula (III):

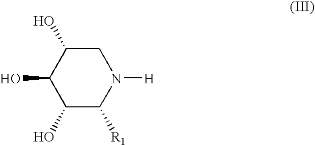

in which R₁ represents an alkyl group as defined above in formula (I) or (I-A), and preferably a nonyl group.

The compounds of formula (III) correspond to compounds of formula (II), in which R₀ represents a hydrogen atom, R₁ represents an alkyl group and R₂, R₃ and R₄ represent an OH group.

A preferred compound of the invention is a compound of the following formula (III-2):

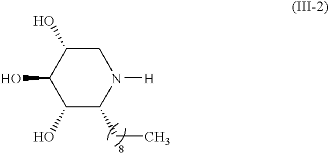

The compounds of formula (III-2) correspond to compounds of formula (III), in which R₁ represents a nonyl group.

The present invention relates to a compound corresponding to the following formula (IV):

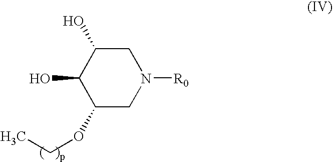

in which:
p represents an integer varying from 0 to 11, and preferably equal to 8,
R₀ represents an alkyl group as defined above, and preferably a nonyl group.

The compounds of formula (IV) correspond to compounds of formula (II), in which R₀ represents an alkyl group, R₁ represents a hydrogen atom, R₂ represents an alkoxy group and R₃ and R₄ represent an OH group.

A preferred compound according to the invention is a compound corresponding to the following formula (IV-2):

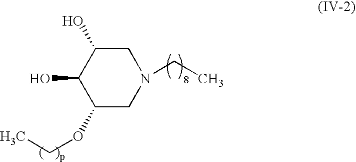

The compounds of formula (IV-2) correspond to compounds of formula (IV), in which R₀ represents a nonyl group.

Another preferred compound according to the invention is a compound corresponding to the following formula (IV-3):

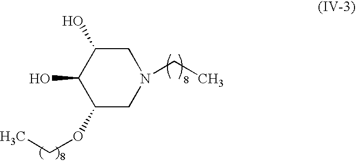

The compound of formula (IV-3) corresponds to a compound of formula (IV-2), in which p is equal to 8.

The present invention also relates to a compound corresponding to the following formula (V):

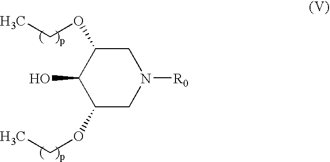

in which:
p represents an integer varying from 0 to 11, and preferably equal to 8,
R₀ represents an alkyl group as defined above, and preferably a nonyl group.

The compounds of formula (V) correspond to compounds of formula (II), in which R₀ represents an alkyl group, R₁ represents a hydrogen atom, R₂ and R₄ represent an alkoxy group and R₃ represents an OH group.

A preferred compound according to the invention is a compound corresponding to the following formula (V-2):

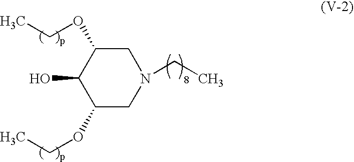

The compounds of formula (V-2) correspond to compounds of formula (V), in which R₀ represents a nonyl group.

Another preferred compound according to the invention is a compound corresponding to the following formula (V-3):

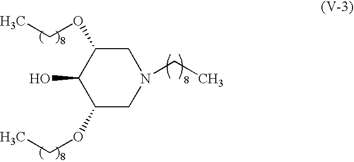

The compound of formula (V-3) corresponds to a compound of formula (V-2), in which p is equal to 8.

The present invention also relates to a pharmaceutical composition comprising a compound of formula (I), (I-A), (I-1), (I-2), (I-3), (I-4), (I-5), (II), (III), (III-2), (IV), (IV-1), (IV-2), (IV-3), (V), (V-1), (V-2) and (V-3) as defined above, in combination with a pharmaceutically acceptable vehicle.

The compounds according to the present invention can be administered by intravenous route, by oral route, by sub-cutaneous, intradermal or epicutaneous route.

The present invention relates to a process for the preparation of a compound of formula (I) as defined above, in which R₁ represents an alkyl group or an n-oxaalkyl group, comprising the following stages:

a) the addition of a organometallic compound, such as a magnesium organic compound or a lithium organic compound, of formula R₁-M, in which R₁ represents an alkyl group or an n-oxaalkyl group as defined above, M represents a metal, preferably Li, or an MgX group in which X represents a halogen atom, preferably Br, on an imine of the following formula (1):

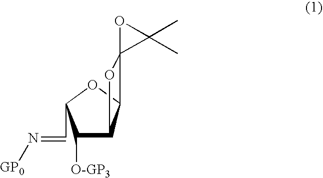

in which:
GP₀ represents a protective group in particular chosen from the allyl, benzyl, p-methoxybenzyl and 2-naphthalenemethyl groups, and preferably represents a benzyl group, and
GP₃ represents a protective group in particular chosen from the allyl, benzyl and 2-naphthalenemethyl groups, and preferably represents a benzyl group, in order to obtain a compound of the following formula (2):

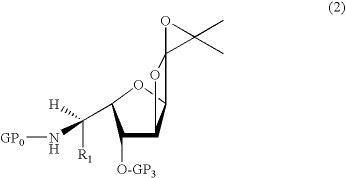

in which GP₀, GP₃ and R₁ are as defined above,

b) the hydrolysis in acid medium of the compound of formula (2) as defined above, followed by an intramolecular reductive amination reaction, in order to obtain a substituted piperidine of the following formula (3):

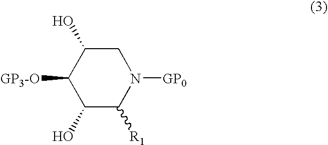

GP₀, GP₃ and R₁ being as defined above,

the compound of formula (3) being, if appropriate, deprotected in order to obtain a compound of formula (III) as defined above,

said compound of formula (III) thus obtained being then optionally subjected to a stage of alkylation of the free amine function, for example by alkylation with an alkyl halide R₀X or by reductive amination with an aldehyde originating from the oxidation of an alcohol of formula R₀OH, R₀ representing an alkyl group or an oxaalkyl group as defined above in the formula, in order to obtain a compound of formula (II) as defined above, in which R₂, R₃ and R₄ represent an OH group,

c) the protection of the free OH functions of the abovementioned compound (3), in order to obtain a substituted piperidine of the following formula (4):

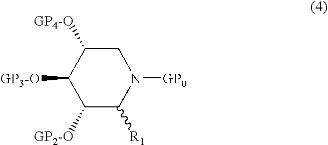

in which:
GP₂ represents a protective group in particular chosen from the acetyl, benzoyl and pivaloyl groups, and preferably represents a benzoyl group,
GP₄ represents a protective group in particular chosen from the trialkylsilyl groups, and preferably represents a t-butyldimethylsilyl group, and
GP₀, GP₃ and R₁ are as defined above,

d) the chemoselective deprotection of one of the GP₂, GP₃ or GP₄ groups, of the compounds of the abovementioned formula (4), in order to obtain respectively a compound of the following formula (5):

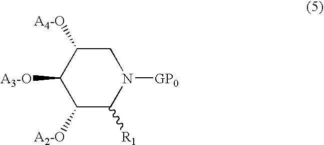

in which:
A₂ represents a hydrogen atom or a protective group GP₂,
A₃ represents a hydrogen atom or a protective group GP₃,
A₄ represents a hydrogen atom or a protective group GP₄,
and only one of the A₂, A₃ and A₄ groups represents H,

GP₀, GP₂, GP₃, GP₄, and R₁ being as defined above,

the compounds of formula (5) including the following compounds:

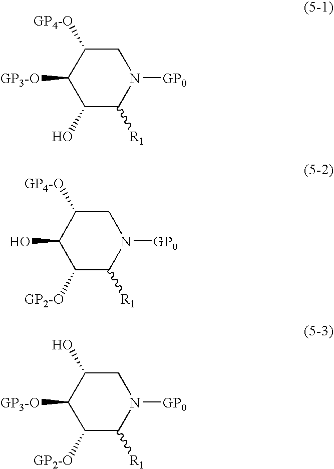

e) reaction of the free hydroxyl function of the abovementioned compounds of formula (5),
either within the framework of the implementation of an alkylation process, for example with an alkyl halide of formula R₅—X, X representing a halogen atom, and R₅ being as defined above, in order to obtain a compound of the following formula (6):

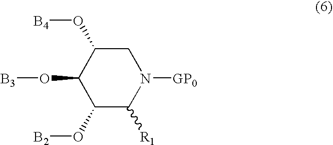

in which:
B₂ represents an R₅ group or a protective group GP₂,
B₃ represents an R₅ group or a protective group GP₃,
B₄ represents an R₅ group or a protective group GP₄,
and only one of the B₂, B₃ and B₄ groups represents R₅,

GP₀, GP₂, GP₃, GP₄, and R₁ being as defined above,

the compounds of formula (6) including the following compounds:

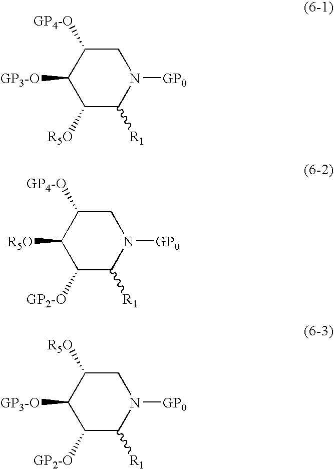

either within the framework of the implementation of an acylation process, for example with an acid chloride of formula R₆COCl, R₆ being as defined above, in order to obtain a compound of the following formula (7):

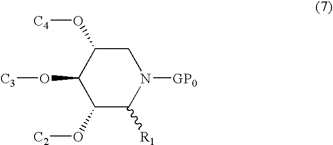

in which:
C₂ represents an COR₆ group or a protective group GP₂,
C₃ represents an COR₆ group or a protective group GP₃,
C₄ represents an COR₆ group or a protective group GP₄,
and only one of the C₂, C₃ and C₄ groups represents COR₆,

GP₀, GP₂, GP₃, GP₄, and R₁ being as defined above,

the compounds of formula (7) including the following compounds:

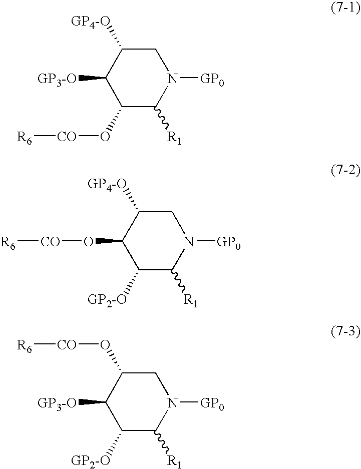

or within the framework of the implementation of a deoxidation process, for example by reaction with Im₂CS then Bu₃SnH, in order to obtain one of the compounds of the following formula (8):

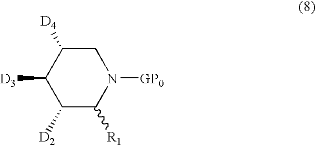

in which:
D₂ represents a hydrogen atom or an OGP₂ group
D₃ represents a hydrogen atom or an OGP₃ group,
D₄ represents a hydrogen atom or an OGP₄ group,
and only one of the D₂, D₃ and D₄ groups represents H,

GP₀, GP₂, GP₃, GP₄, and R₁ being as defined above,

the compounds of formula (8) including the following compounds:

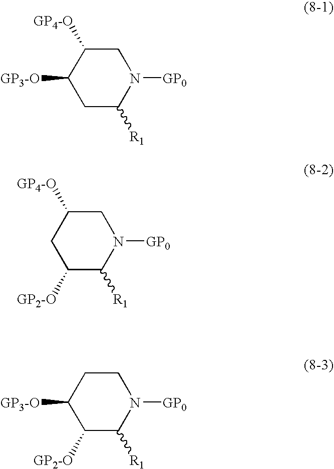

either within the framework of the implementation of a configuration inversion process, for example by the implementation of the Swern reaction, followed by reduction with a boron hydride, in order to obtain one of the compounds of the following formulae (9-1), (9-2) or (9-3):

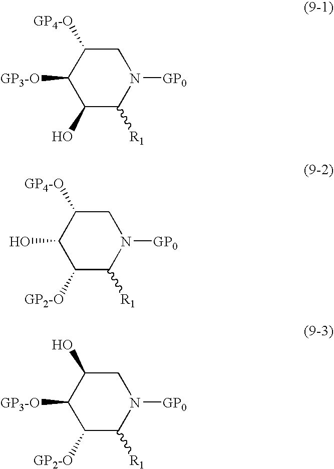

GP₀, GP₂, GP₃, GP₄, and R₁ being as defined above,

the abovementioned compounds of formulae (6), (7), (8), (9-1), (9-2) and (9-3) being able to be deprotected in order to obtain respectively the compounds of the following formulae (I-6), (I-7), (I-8), (I-9-1), (I-9-2) and (I-9-3), corresponding to compounds of formula (I) as defined above:

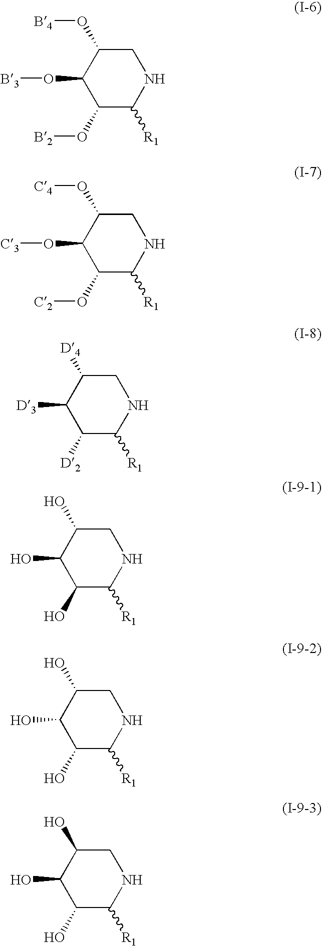

in which:
B′₂ represents an R₅ group or a hydrogen atom,
B′₃ represents an R₅ group or a hydrogen atom,
B′₄ represents an R₅ group or a hydrogen atom,
and only one of the B′₂, B′₃ and B′₄ groups represents R₅,
C′₂ represents a COR₆ group or a hydrogen atom,
C′₃ represents a COR₆ group or a hydrogen atom,
C′₄ represents a COR₆ group or a hydrogen atom,
and only one of the C′₂, C′₃ and C′₄ groups represents COR₆,
D′₂ represents a hydrogen atom or an OH group,
D′₃ represents a hydrogen atom or an OH group,
D′₄ represents a hydrogen atom or an OH group,
and only one of the OF₂, OF₃ and OF₄ groups represents H,
   the compounds thus obtained of formulae (I-6), (I-7), (I-8), (I-9-1), (I-9-2) and (I-9-3) being able to then be optionally subjected to a stage of alkylation of the free amine function, for example by alkylation with an alkyl halide R₀X or by reductive amination with an aldehyde originating from the oxidation of an alcohol of formula R₀OH, R₀ representing an alkyl group or an oxaalkyl group as defined above, in order to obtain the compounds of the following formulae (I-6-a), (I-7-a), (I-8-a), (I-9-1-a), (I-9-2-a) and (I-9-3-a), corresponding to compounds of formula (I) as defined above:

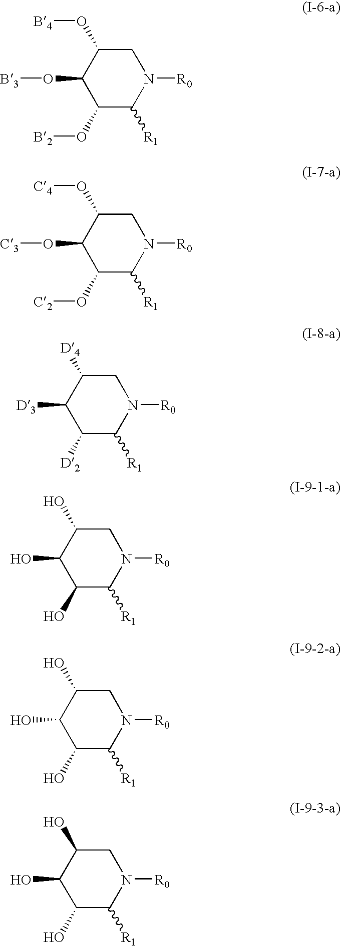

B′₂, B′₃, B′₄, C′₂, C′₃, C′₄, D′₂, D′₃, D′₄, R₀ and R₁ being as defined above,

f) and, optionally, the regioselective deprotection of one of the remaining protective groups GP₂, GP₃ or GP₄, of the abovementioned compounds of formula (6), (7), (8), (9-1), (9-2) or (9-3) in order to obtain a deprotected free hydroxyl function, and the reaction of this free hydroxyl function, as described previously in stage e), either within the framework of the implementation of an alkylation process, or within the framework of the implementation of an acylation process, or within the framework of the implementation of a deoxidation process, or within the framework of the implementation of a configuration inversion process, and an optional deprotection stage in order to obtain compounds of formula (I) as defined above, optionally followed by a stage of alkylation of the free amine function, for example by alkylation with an alkyl halide R₀X or by reductive amination with an aldehyde originating from the oxidation of an alcohol of formula R₀OH, R₀ representing an alkyl group or an oxaalkyl group as defined above in formula (I), in order to obtain compounds of formula (I) in which R₀ is different from a hydrogen atom,

stage f) being able to be optionally repeated if the compound obtained of formula (I) also contains a free hydroxyl function.
The process of the invention can be represented for example according to the following diagram:

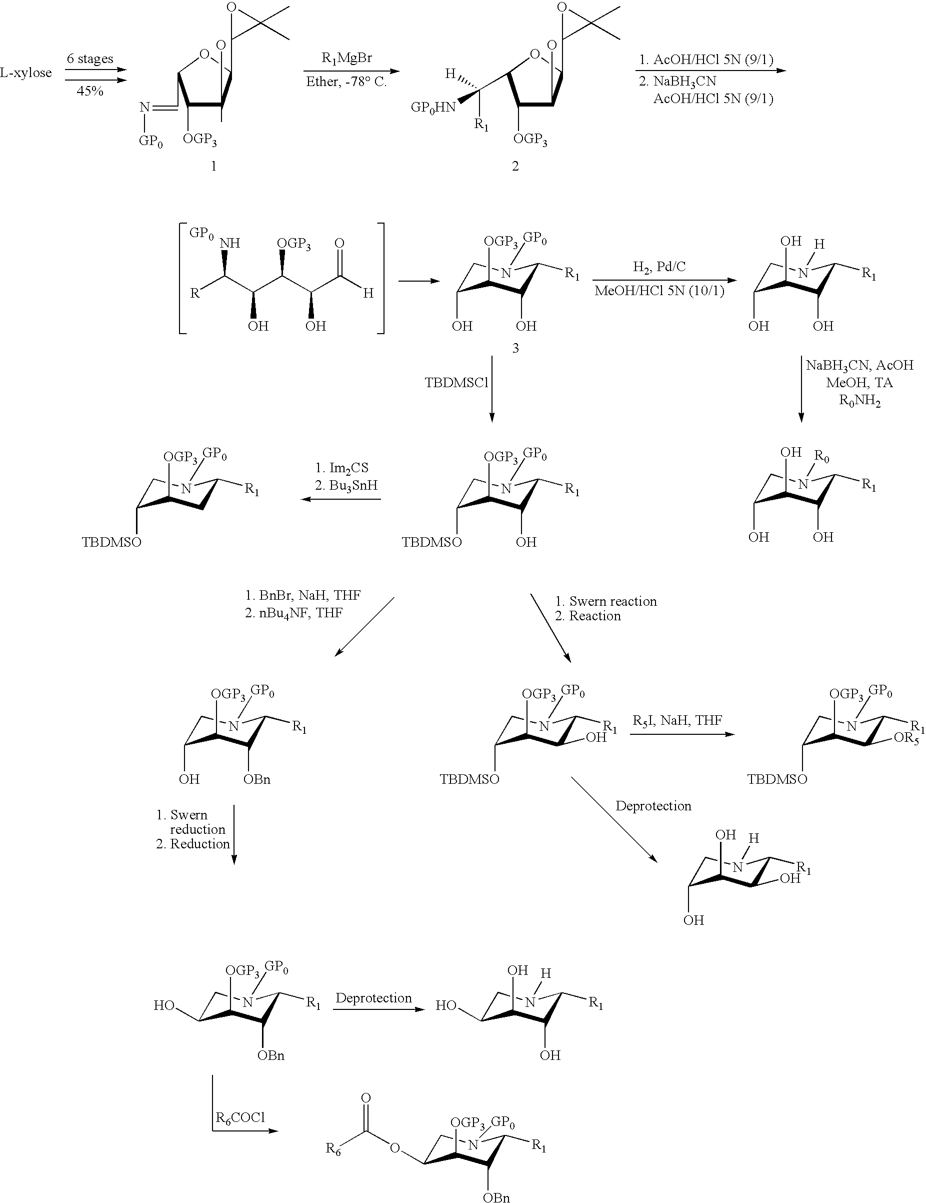

General Operating Method

Starting with a 1,2-O-isopropylidene L-xylofuranose derivative selectively protected at position 3 (Bordier, A.; Compain, P.; Martin, O. R.; Ikeda, K.; Asano, N. First Stereocontrolled Synthesis and Biological Evaluation of 1,6-Dideoxy-L-nojirimycin. Tetrahedron. Asymmetry 2003, 14, 47-51), for example with a benzyl, an oxidation reaction is carried out at position 5 in order to obtain the corresponding aldehyde which is then used in order to form the corresponding imine by reaction with a primary amine, for example benzylamine. An organometallic reagent, such as for example a magnesium organic compound or a lithium organic compound is then added to this imine, optionally in the presence of Lewis acid, then the hydrolysis of the isopropylidene is carried out in acid medium followed by an intramolecular reductive amination reaction, in the presence of NaBH₃CN for example, in order to produce a substituted piperidine of general formula (3) as mentioned above.

The compound of general formula (3) can then be deprotected in order to produce the corresponding piperidinols (R₀=GP₃═H). The amine function of these derivatives can then be alkylated by alkylation with an alkyl halide or by reductive amination by reaction with an aldehyde, in the presence of NaBH₃CN for example.

Alternatively, the compound of general formula (3) is regioselectively protected at position 4, for example in the form of a silyl ether. The remaining secondary alcohol is then protected in orthogonal manner with, for example, a benzoate or acetate group in order to produce a substituted piperidine of general formula (4) as defined above.

Each of the 3 groups GP₄, GP₃ and GP₂ is chemoselectively deprotected in order to produce the corresponding C4, C3 and C2 secondary hydroxyl respectively. This hydroxyl is either alkylated, for example by use of an alkyl halide in the presence of NaH, or acylated for example by use of an acid chloride, or deoxidized, for example by reaction with Im₂CS then Bu₃SnH, or its configuration is inverted, for example by a 2-stage strategy: oxidation in ketone then reduction with a hydride such as NaBH₄ or L-selectride. The regioselective deprotection of one of the other two secondary hydroxyls remaining in the compounds obtained makes it possible to obtain a free alcohol which can be either alkylated, for example by use of an alkyl halide in the presence of NaH, or acylated, for example by use of an acid chloride, or deoxidized, for example by reaction with Im₂CS then nBu₃SnH, or its absolute configuration is inverted, for example by a 2-stage strategy: oxidation in ketone then reduction with a hydride such as NaBH₄ or L-selectride. The compounds obtained by the abovementioned different synthesis routes are then deprotected in a standard manner.

The present invention also relates to a process for the preparation of a compound of formula (III) as defined above,

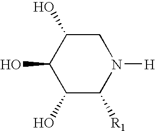

comprising the following stages:

a) the addition of an organometallic compound, such as a magnesium organic compound or a lithium organic compound, of formula R₁-M, in which R₁ represents an alkyl group or an n-oxaalkyl group as defined in formula (III), M represents a metal, preferably Li, or an MgX group in which X represents a halogen atom, preferably Br, on an imine of the following formula (1):

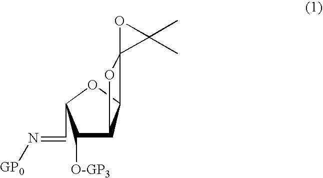

in which:
GP₀ represents a protective group in particular chosen from the allyl, benzyl, p-methoxybenzyl and 2-naphthalenemethyl groups, and preferably represents a benzyl group, and
GP₃ represents a protective group in particular chosen from the allyl, benzyl and 2-naphthalenemethyl groups, and preferably represents a benzyl group,
   in order to obtain a compound of the following formula (2):

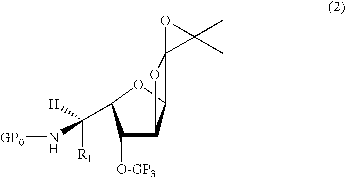

in which GP₀, GP₃ and R₁ are as defined above, and

b) the hydrolysis in acid medium of the compound of formula (2) as defined above, followed by an intramolecular reductive amination reaction, in order to obtain a substituted piperidine of the following formula (3):

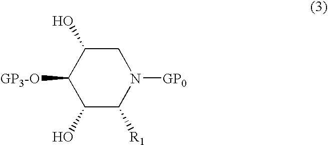

GP₀, GP₃ and R₁ being as defined above,

the compound of formula (3) being deprotected in order to obtain a compound of formula (III) as defined above.

The present invention also relates to a process for the preparation of a compound of formula (I) as defined above, in which R₁ represents a hydrogen atom, comprising the following stages:

a) the reaction of a compound of the following formula (10):

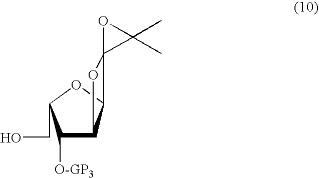

in which GP₃ represents a protective group in particular chosen from the allyl, benzyl and 2-naphthalenemethyl groups, and preferably represents a benzyl group,
with a reagent containing an activating group Y, in order to introduce said activating group at position 5 of the compound of formula (10), in order to obtain the compound of the following formula (11):

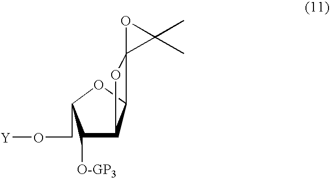

Y—O representing a leaving group, in which Y is an activating group in particular chosen from the groups, p-toluenesulphonyl and trifluoromethanesulphonyl mesyl, and preferably being a mesyl group,

b) the substitution of the compound of the abovementioned formula (11) by a primary amine R₀NH₂, R₀ representing an alkyl or oxaalkyl group as defined above, in order to obtain a compound of the following formula (12):

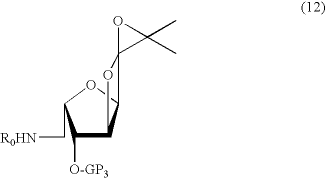

GP₃ being as defined above,

c) the hydrolysis in acid medium of the compound of formula (12) as defined above, followed by an intramolecular reductive amination reaction, in order to obtain a substituted piperidine of the following formula (13):

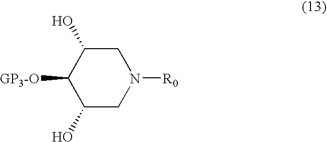

GP₃ and R₀ being as defined above,

d) the reaction of the compound of the abovementioned formula (13) with a compound of formula R₅X, R₅ being as defined above and X representing a halogen atom, or with an acid chloride R₆COCl, R₆ being as defined above, in order to obtain one of the following compounds (14-1) or (14-2):

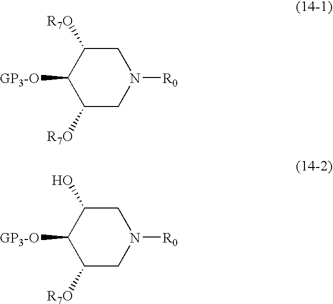

R₇ representing a COR₆ group or a R₅ group as defined above,
GP₃ and R₀ being as defined above,

the compounds of formulae (14-1) and (14-2) being in particular separated by silica gel chromatography,

the compound of formula (14-2) being, if appropriate, deprotected in order to obtain a compound of the following formula (I):

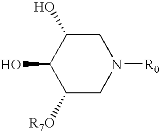

R₇ and R₀ being as defined above,

e) the regioselective deprotection of the OGP₃ group of the compound of formula (14-1) in order to obtain the compound of the following formula (14-3) containing a free hydroxyl function and corresponding to a compound of formula (I):

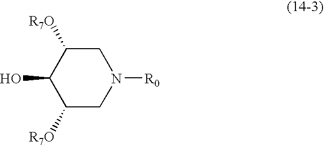

R₇ and R₀ being as defined above,

f) the reaction of the free hydroxyl function of the abovementioned compounds of formula (14-2) and (14-3),
either within the framework of the implementation of an alkylation process, for example with an alkyl halide of formula R₅—X, X representing a halogen atom, and R₅ being as defined above, in order to obtain a compound of the following formula (15-2) or (15-3) respectively:

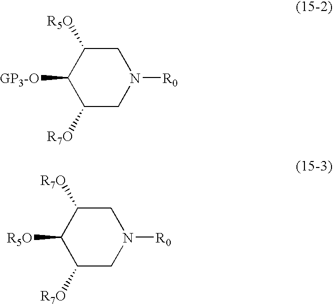

GP₃, R₅, R₇ and R₀ being as defined above,

the compound of formula (15-2) then being deprotected in order to obtain a compound of the following formula (1):

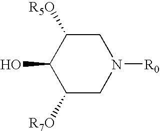

R₅, R₇ and R₀ being as defined above,
or within the framework of the implementation of an acylation process, for example with an acid chloride of formula R₆COCl, R₆ being as defined above, in order to obtain respectively a compound of the following formula (16-2) or (16-3):

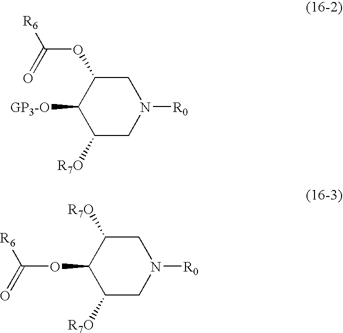

GP₃, R₆, R₇ and R₀ being as defined above,

the compound of formula (16-2) then being deprotected in order to obtain a compound of the following formula (I):

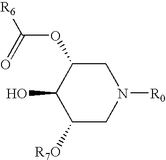

R₆, R₇ and R₀ being as defined above,
or within the framework of the implementation of a deoxidation process, for example by reaction with Im₂CS then Bu₃SnH, in order to obtain a compound of the following formula (17-2) or (17-3) respectively:

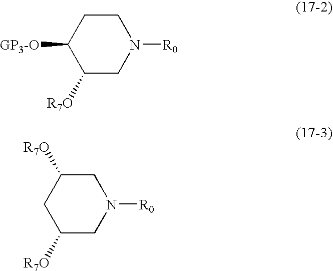

GP₃, R₇ and R₀ being as defined above,

the compound of formula (17-2) then being deprotected in order to obtain a compound of the following formula (I):

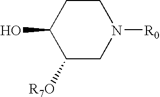

R₇ and R₀ being as defined above,
or within the framework of the implementation of a configuration inversion process, for example by the implementation of the Swern reaction, followed by a reduction with a boron hydride, in order to obtain a compound of the following formula (18-2) or (18-3) respectively:

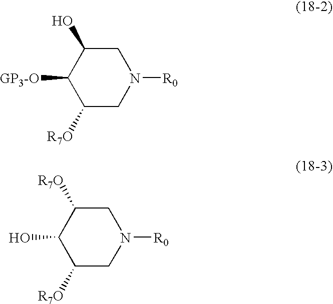

GP₃, R₇ and R₀ being as defined above,

the compound of formula (18-2) then being deprotected in order to obtain a compound of the following formuula (1):

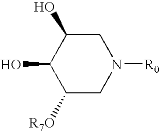

R₇ and R₀ being as defined above.
The process of the invention can be represented for example according to the following diagram:

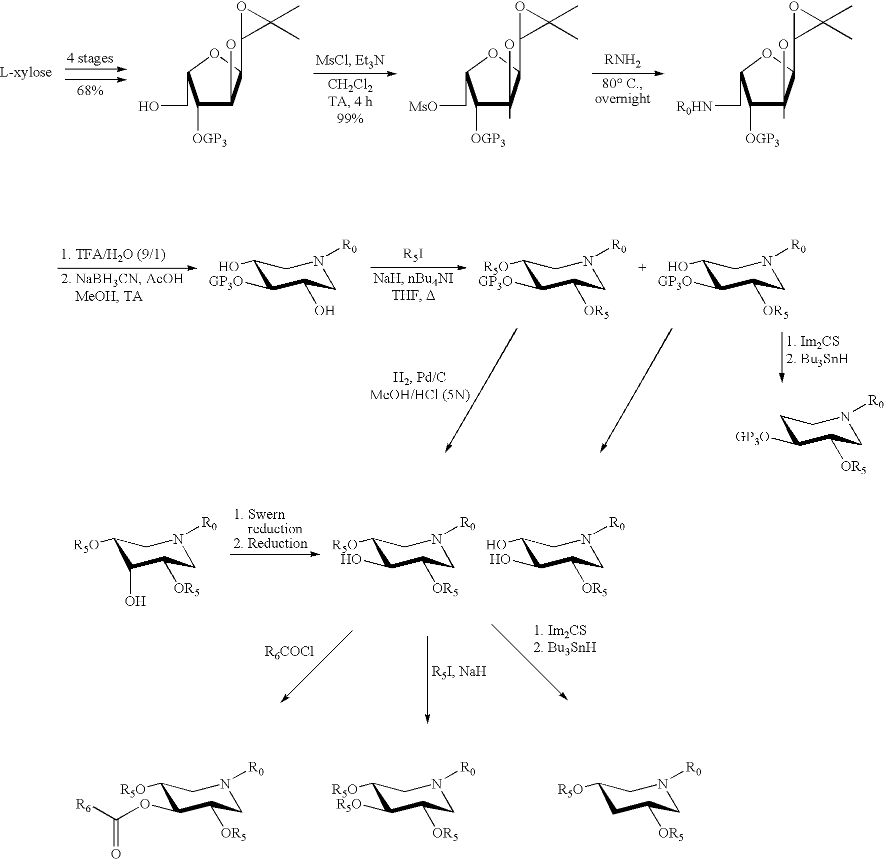

### General Operating Method

Starting with a 1,2-O-isopropylidene L-xylofuranose derivative selectively protected at position 3 (Bordier, A.; Compain, P.; Martin, O. R.; Ikeda, K.; Asano, N. First Stereocontrolled Synthesis and Biological Evaluation of 1,6-Dideoxy-L-nojirimycin. Tetrahedron. Asymmetry 2003, 14, 47-51), for example with a benzyl, a leaving group is introduced at position 5 for example a mesyl group by action of mesyl chloride in the presence of triethylamine. The leaving group is then substituted by a primary amine in order to produce the corresponding secondary amine. The hydrolysis of the isopropylidene in acid medium followed by a reductive amination reaction, in the presence of NaBH₃CN for example, makes it possible to obtain a substituted piperidine of general formula (13) as described previously.

The compound of general formula (13) is then either alkylated, for example by use of an alkyl halide in the presence of NaH, or acylated for example by use of an acid chloride in order to produce the corresponding mono- and disubstituted piperidines at positions 2 and 4 in order to produce the compound of general formula (14-2) and the compound of general formula (14-1) respectively. The two compounds are then separated by silica gel chromatography.

The C4 hydroxyl of the compound of general formula (14-2) is either alkylated, for example by use of an alkyl halide in the presence of NaH, or acylated for example by use of an acid chloride, or deoxidized, for example by reaction with Im₂CS then nBu₃SnH, or its absolute configuration is inverted, for example by a 2-stage strategy: oxidation in ketone then reduction with a hydride such as NaBH₄ or L-selectride, or protected in orthogonal manner for example in the form of a silyl ether.

The regioselective deprotection of the C3 hydroxyl of the compound of general formula (14-1) makes it possible to obtain a free alcohol which can be either alkylated, for example by use of an alkyl halide in the presence of NaH, or acylated for example by use of an acid chloride, or deoxidized, for example by reaction with Im₂CS then nBu₃SnH, or its absolute configuration is inverted, for example by a 2-stage strategy: oxidation in ketone then reduction with a hydride such as NaBH₄ or L-selectride.

The compounds obtained by the abovementioned different synthesis routes are then deprotected in standard manner.

Part of the process is described in the following article: Bordier, A.; Compain, P.; Martin, O. R.; Ikeda, K.; Asano, N. First Stereocontrolled Synthesis and Biological Evaluation of 1,6-Dideoxy-L-nojirimycin. Tetrahedron: Asymmetry 2003, 14, 47-51.

The present invention also relates to a process for the preparation of a compound of formula (IV) as defined above,

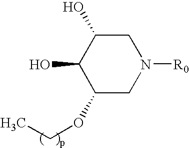

comprising the following stages:

a) the reaction of a compound of the following formula (10):

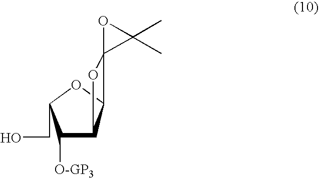

in which GP₃ represents a protective group in particular chosen from the allyl, benzyl and 2-naphthalenemethyl groups, and preferably represents a benzyl group,

with a reagent containing an activating group Y, in order to introduce said activating group at position 5 of the compound of formula (10), in order to obtain the compound of the following formula (11):

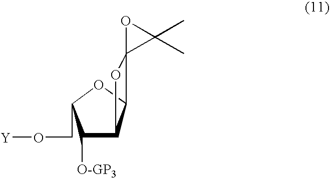

Y—O representing a leaving group, in which Y is an activating group in particular chosen from the mesyl, p-toluenesulphonyl and trifluoromethanesulphonyl groups, and preferably being a mesyl group,

b) the substitution of the compound of the abovementioned formula (11) by a primary amine R₀NH₂, R₀ representing an alkyl or oxaalkyl group as defined above, in order to obtain a compound of the following formula (12):

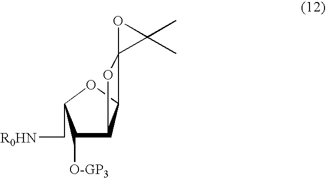

GP₃ being as defined above,
c) the hydrolysis in acid medium of the compound of formula (12) as defined above, followed by an intramolecular reductive amination reaction, in order to obtain a substituted piperidine of the following formula (13):

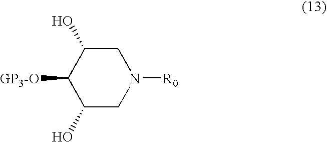

GP₃ and R₀ being as defined above,

d) the reaction of the compound of the abovementioned formula (13) with a compound of formula R₅X, R₅ being as defined above and representing in particular a —(CH₂)ₚ—CH₃ group and X representing a halogen atom, in order to obtain one of the following compounds (14-1-1) or (14-2-1):

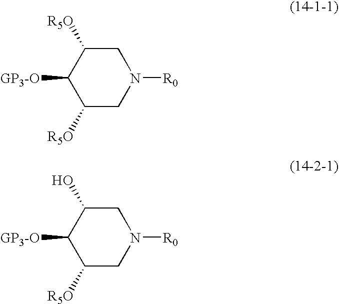

GP₃, R₅ and R₀ being as defined above,

the compounds of formulae (14-1-1) and (14-2-1) being in particular separated by silica gel chromatography, and

the compound of formula (14-2-1) being deprotected in order to obtain a compound of the following formula (IV)

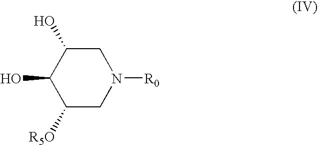

R₅ and R₀ being as defined above.

The present invention also relates to a process for the preparation of a compound of formula (V) as defined above,

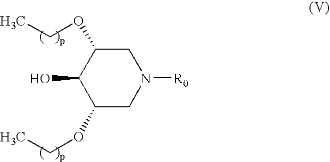

comprising the following stages:

a) the reaction of a compound of the following formula (10):

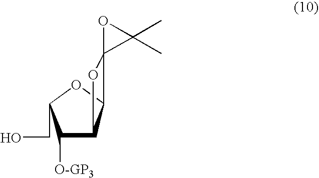

in which GP₃ represents a protective group in particular chosen from the allyl, benzyl and 2-naphthalenemethyl groups, and preferably represents a benzyl group,

with a reagent containing an activating group Y, in order to introduce said activating group at position 5 of the compound of formula (10), in order to obtain the compound of the following formula (11):

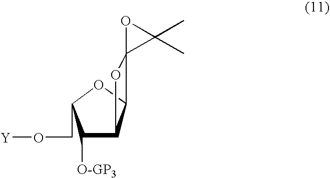

Y—O representing a leaving group, in which Y is an activating group in particular chosen from the mesyl, p-toluenesulphonyl and trifluoromethanesulphonyl groups, and preferably being a mesyl group,

b) the substitution of the compound of the abovementioned formula (11) by a primary amine R₀NH₂, R₀ representing an alkyl or oxaalkyl group as defined above, in order to obtain a compound of the following formula (12):

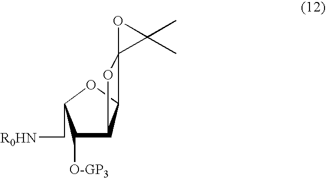

GP₃ being as defined above,

c) the hydrolysis in acid medium of the compound of formula (12) as defined above, followed by an intramolecular reductive amination reaction, in order to obtain a substituted piperidine of the following formula (13):

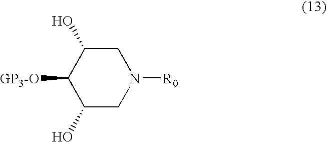

GP₃ and R₀ being as defined above,

d) the reaction of the compound of the abovementioned formula (13) with a compound of formula R₅X, R₅ being as defined above and representing in particular a —(CH₂)ₚ—CH₃ group and X representing a halogen atom, in order to obtain one of the following compounds (14-1-1) or (14-2-1):

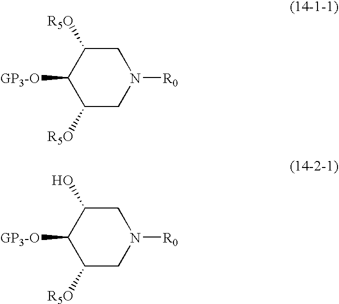

GP₃, R₅ and R₀ being as defined above,

the compounds of formulae (14-1-1) and (14-2-1) being in particular separated by silica gel chromatography, and

e) the regioselective deprotection of the OGP₃ group of the compound of formula (14-1-1) in order to obtain the compound of the following formula (14-3-1) containing a free hydroxyl function and corresponding to a compound of formula (V).

The invention is further illustrated by means of the detailed description which follows of the obtaining of preferred compounds of the invention, and of their biological properties.

Within the framework of works on the synthesis of C-glycosylated iminosugars, effective synthesis methodologies have been developed for obtaining derivatives of iminosugars carrying at the pseudoanorneric position aglycone mimics of certain glycosides. In particular the addition of a substituted or unsubstituted alkyl chain makes it possible to markedly increase the selectivity of the iminoalditols as glycosidase inhibitors (Godin, G.; Compain, P; Martin, O. R.; Ikeda, K.; Asano, N. α-1-C-Alkyl-1-deoxynojirimycin derivatives as potent and selective inhibitors of intestinal isomaltase: remarkable effect of the alkyl chain length on glycosidase inhibitory profile. Bioorg. Med. Chem. Lett. 2004, 14, 5991-5995). By a study of structure-activity relationship, it has been possible to demonstrate α-1-C-nonyliminoxylitol 1′a as the most powerful and the most selective currently known inhibitor of human β-glucocerebrosidase (Kᵢ=2 nM). Compound 1′a (α-1-C-nonyl-XYL) is thus 150 times more active than N-nonyl-1-deoxynojirimycin 2′ (N-nonyl-DNJ) described by Wong's group in 2002 (Sawkar. A. R.; Cheng, W.-C.; Beutler, E.; Wong, C.-H.; Balch, W. E.; Kelly, J. W. Chemical chaperones increase the cellular activity of N370S β-glucosidase: A therapeutic strategy for Gaucher's disease. Proc. Natl. Acad. Sci. USA 2002, 99, 15428). Cell tests carried out on fibroblasts originating from patients suffering from Gaucher's disease of type 1, 2 or 3 have shown that the use of very low quantities of 1′a made it possible to double the residual enzymatic activity of human β-glucocerebrosidase.

Compound 1′a corresponds to a compound of formula (III-2) as defined above, and is a compound of formula (I) in which R₀ represents a hydrogen atom, R₁ represents a nonyl group and R₂, R₃ and R₄ represent an OH group.

### EXPERIMENTAL PART

I—Synthesis of the Iminosugars

The general synthesis strategy used to obtain α-1-C-alkyl-iminoxylitols is described in Diagram 1 (see below). The starting compound 3′ is synthesized following a methodology published in Bordier, A.; Compain, P.; Martin, O. R.; Ikeda, K.; Asano, N. First Stereocontrolled Synthesis and Biological Evaluation of 1,6-Dideoxy-L-nojirimycin. Tetrahedron: Asymmetry 2003, 14, 47-51. This general synthesis strategy makes it possible to obtain α-1-C-alkyl-iminoxylitols with overall yields comprised between 27% and 43% in 9 stages starting with L-xylose.

The first stage relates to the addition of a magnesium organic compound to the imine 3′ in ether which leads to the formation of the C-1 amines 4′ of R configuration in the form of a single diastereoisomer. The deprotection of the acetals 4′ in acetic acid in the presence of concentrated hydrochloric acid, followed by an intramolecular reductive amination reaction by adding NaBH₃CN, makes it possible to obtain the expected C-glycosylated iminosugars 5′. These compounds are then easily deprotected under a hydrogen atmosphere in the presence of palladium on carbon in order to produce the α-1-C-alkyl-iminoxylitols 1′.

Protocols

General Remarks

The reactions requiring rigorously anhydrous conditions were carried out with glassware placed in an oven (140° C.), then cooled down in a desiccator containing calcium chloride. A flow of argon dried by filtering on three levels (soda, calcium chloride, soda) is delivered by a double ramp. The diethyl ether was distilled on sodium and benzophenone.

The purifications by column chromatography were carried out on Merck 40-70 μm “flash” silica gel (230-400 mesh) under pressure of nitrogen.

1) Preparation of the Iminoxylitols 1′a and 1′b

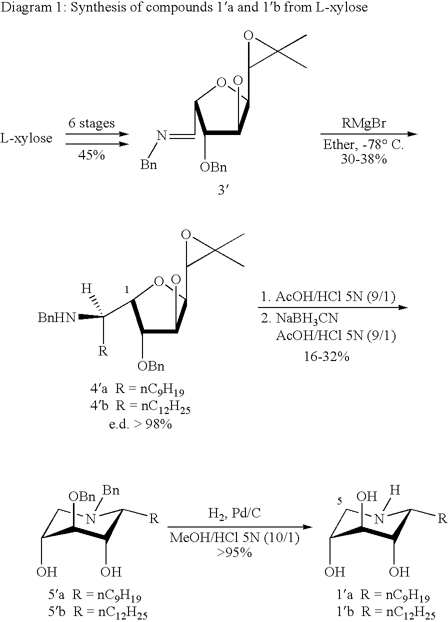

General Operating Method for Obtaining the Aminated Compounds 4′a and 4′b

The imine 3′ is solubilized in freshly distilled diethyl ether (0.06 M), under a current of argon and at −78° C. A 1M solution in diethyl ether of 4 equivalents of nonyl magnesium bromide (in order to obtain the amine 4′a) or of dodecyl magnesium bromide (in order to obtain the amine 4′b) is then added dropwise and the reaction medium is stirred for 3 hours at −78° C. A saturated ammonium chloride solution is added dropwise, the reaction being very exothermic. After separation of the phases, the organic phase is dried over magnesium sulphate and concentrated under reduced pressure. The residue obtained is purified by silica gel chromatography using the eluent toluene/ethyl acetate (5/1) in order to obtain the desired product.

### Characteristics of Compound 4′a

Yield: 38%

Appearance: orange oil

Rf: 0.15 (toluene/ethyl acetate 4/1)

HRMS (ESI): m/z 496.3423 [M+H]⁺ (theoretical 496.3427)

NMR ¹H (CDCl₃), δ(ppm):

0.88 (t, 3H, J=6.9 Hz); 1.25 (m, 16H); 1.32 (s, 3H); 1.48 (s, 3H); 3.11 (m, 1H); 3.79 (s, 2H); 3.86 (d, 1H, J=2.8 Hz); 4.11 (dd, 1H, J=2.8 and 9.1 Hz); 4.43 (d, 1H, J=11.6 Hz); 4.63 (d, 1H, J=4.1 Hz); 4.68 (d, 1H, J=11.6 Hz); 5.94 (d, 1H, J=3.8 Hz); 7.20-7.34 (m, 10H).

NMR ¹³C (CDCl₃), δ(ppm):

14.3; 22.8; 25.4; 26.4; 26.8; 29.4; 29.7; 29.8; 30.1; 30.3; 32.1; 51.3; 55.7; 71.7; 81.9; 82.0; 82.8; 104.8; 111.5; 126.8; 128.1; 128.2; 128.4; 128.5; 128.6; 128.7; 137.3; 141.1

### Characteristics of Compound 4′b

Yield: 30%

Appearance: orange oil

Rf: 0.3 (toluene/ethyl acetate 4/1)

HRMS (ESI): m/z 538.3897 [M+H]⁺ (theoretical 538.3896)

NMR ¹H (CDCl₃), δ(ppm):

0.88 (t, 3H, J=6.8 Hz); 1.26 (m, 22H); 1.32 (s, 3H); 1.48 (s, 3H); 3.11 (m, 1H); 3.79 (s, 2H); 3.86 (d, 1H, J=2.8 Hz); 4.11 (dd, 1H, J=2.8 and 9.1 Hz); 4.43 (d, 1H, J=11.7 Hz); 4.63 (d, 1H, J=3.6 Hz); 4.68 (d, 1H, J=11.7 Hz); 5.94 (d, 1H, J=3.9 Hz); 7.19-7.33 (m, 10H)

NMR ¹³C (CDCl₃), δ(ppm):

14.2; 22.8; 25.4; 26.4; 26.8; 29.5; 29.7; 29.8; 30.1; 30.3; 32.0; 51.3; 55.7; 71.7; 81.87; 81.94; 82.8; 104.7; 111.5; 126.8; 128.06; 128.1; 128.36; 128.5; 128.55; 137.3; 141.1

General Operating Method for Obtaining the Iminosugars 5′a and 5′b

The amines 4′ are solubilized in a mixture of acetic acid (0.2 M) and hydrochloric acid (5N)(9/1). The reaction medium is stirred at ambient temperature for 5 hours and 30 minutes. Sodium cyanoborohydride (9 eq) is then added and the reaction medium is stirred for 4.5 days at ambient temperature. A saturated solution of sodium carbonate and a solution of soda (2M) are added, at 0° C., to neutralize the reaction medium followed by extraction 3 times with ethyl acetate, the organic phase is dried over magnesium sulphate and concentrated under reduced pressure. The residue obtained is purified by silica gel chromatography using the eluent toluene/ethyl acetate (10/1) in order to obtain the desired product.

### Characteristics of Compound 5′a

Yield: 16%

Appearance: white solid

Rf: 0.2 (toluene/ethyl acetate 4/1+1% Et₃N)

MS: m/z 440.0 [M+H]⁺ (theoretical 439.6)

[α]_{D}−17.7 (c=1, CHCl₃)

NMR ¹H (CDCl₃), δ(ppm):

0.88 (t, 3H, J=6.6 Hz); 1.20-1.40 (m, 14H); 1.63 (m, 2H); 2.64 (m, 2H); 2.79 (m, 1H); 3.48 (d, 1H, J=13.5 Hz); 3.54 (d, 1H, J=6.3 Hz); 3.78 (m, 1H); 3.86 (m, 1H); 3.93 (d, 1H, J=13.5 Hz); 4.69 (d, 1H, J=11.9 Hz); 4.75 (d, 1H, J=12.2 Hz); 7.23-7.36 (m, 10H)

NMR ¹³C (CDCl₃), δ(ppm):

14.3; 22.8; 26.4; 27.6; 29.5; 29.7; 30.2; 32.1; 52.5; 57.8; 61.5; 69.3; 70.5; 73.6; 80.8; 127.1; 127.8; 128.0; 128.5; 128.7; 138.6; 139.7

### Characteristics of Compound 5′b

Yield: 32%

Appearance: white solid

Rf: 0.1 (toluene/ethyl acetate 10/1+1% Et₃N)

HRMS (ESI): m/z 482.3629 [M+H]⁺ (theoretical 482.36342)

NMR ¹H (CDCl₃), δ(ppm):

0.88 (t, 3H, J=6.6 Hz); 1.20-1.40 (m, 20H); 1.63 (m, 2H); 2.65 (m, 2H); 2.78 (m, 1H); 3.48 (d, 1H, J=9.4 Hz); 3.53 (d, 1H, J=6.6 Hz); 3.77 (m, 1H); 3.86 (m, 1H); 3.92 (d, 1H, J=13.5 Hz); 4.68 (d, 1H, J=11.9 Hz); 4.73 (d, 1H, J=12.2 Hz); 7.22-7.33 (m, 10H)

NMR ¹³(CDCl₃), δ(ppm):

14.3; 22.8; 26.4; 27.6; 29.5; 29.8; 30.2; 32.1; 52.5; 57.8; 61.4; 69.2; 70.4; 73.6; 80.7; 127.2; 127.8; 128.0; 128.5; 128.69; 128.71; 138.6; 139.6

General Operating Method for Obtaining the Iminosugars 1′a and 1′b

The iminosugars 5′ are solubilized, at ambient temperature and under a current of argon, in a mixture of methanol (0.01 M) and hydrochloric acid (5N) (10/1). Then palladium on activated carbon (10 mole %) is added to the reaction medium. The solution is then placed under vacuum, then under hydrogen. The mixture is stirred for 24 hours at ambient temperature, then filtered on a millipore filter, rinsed with methanol and concentrated under reduced pressure in order to produce the expected crude product. This compound is purified by an Amberlyst 15 [H⁺] ion-exchange resin column (eluent: 1M aqueous solution of ammonium hydroxide).

### Characteristics of Compound 1′a

Yield: quantitative

Appearance: white solid

MS: m/z 260.0 [M+H]⁺ (theoretical 259.4)

NMR ¹H (CD₃OD), δ(ppm):

0.89 (t, 3H, J=7.0 Hz); 1.29-1.38 (m, 14H); 1.44 (m, 1H); 1.56 (m, 1H); 2.78 (dd, 1H, J=3.7 and 13.5 Hz); 2.88 (dt, 1H, J=2.3 and 7.3 Hz); 3.03 (dd, 1H, J=2.7 and 13.5 Hz); 3.53 (m, 2H); 3.76 (t, 1H, J=4.0 Hz)

NMR ¹³C (125 MHz, CD₃OD), δ(ppm):

14.5; 23.8; 27.2; 30.5; 30.75; 30.84; 30.93; 30.98; 33.1; 47.5; 55.6; 70.7; 71.4; 71.9

### Characteristics of Compound 1′b

Yield: quantitative

Appearance: white solid

HRMS (FAB): m/z 302.2697 [M+H]⁺ (theoretical 302.2695)

[α]_{D}−19.0 (c=0.4, MeOH]

NMR ¹H (CD₃OD), δ(ppm):

0.90 (t, 3H, J=6.9 Hz); 1.27-1.35 (m, 20H); 1.43 (m, 1H); 1.54 (m, 1H); 2.76 (dd, 1H, J=3.7 and 13.5 Hz); 2.88 (dt, 1H, J=2.3 and 7.3 Hz); 3.02 (dd, 1H, J=2.8 and 13.5 Hz); 3.54 (m, 2H); 3.76 (t, 1H, J=4.6 Hz)

NMR ¹³C (CD₃OD), δ(ppm):

14.5; 23.8; 27.2; 30.5; 30.7; 30.78; 30.8 (2×C); 30.83 (2×C); 30.9; 33.1; 47.5; 55.6; 70.6; 71.5; 71.8

2) Preparation of the Iminoxylitols 10′ and 11′

The first stages of the preparation of the iminoxylitols 10′ and 11′ involve a synthesis strategy described by Bordier, A.; Compain, P.; Martin, O. R.; Ikeda, K.; Asano, N. Tetrahedron. Asymmetry 2003, 14, 47-51. The octylamine group is introduced in two stages starting with 3-O-benzyl-1,2-O-isopropylidene-α-L-xylofuranose via the mesylate 6′. As within the framework of the synthesis of the compounds 1′, the key stage of this strategy involves deprotection in acid medium of the acetal function of 7′ followed by an intramolecular reductive amination reaction by addition of NaBH₃CN in order to produce the expected diol 8′. A non-regioselective alkylation reaction leads to the tri-alkylated compound 9′a and di-alkylated compound 9′b. These derivatives are separated on silica gel and deprotected in order to produce the corresponding iminoxylitols 10′ and 11′ with good yields.

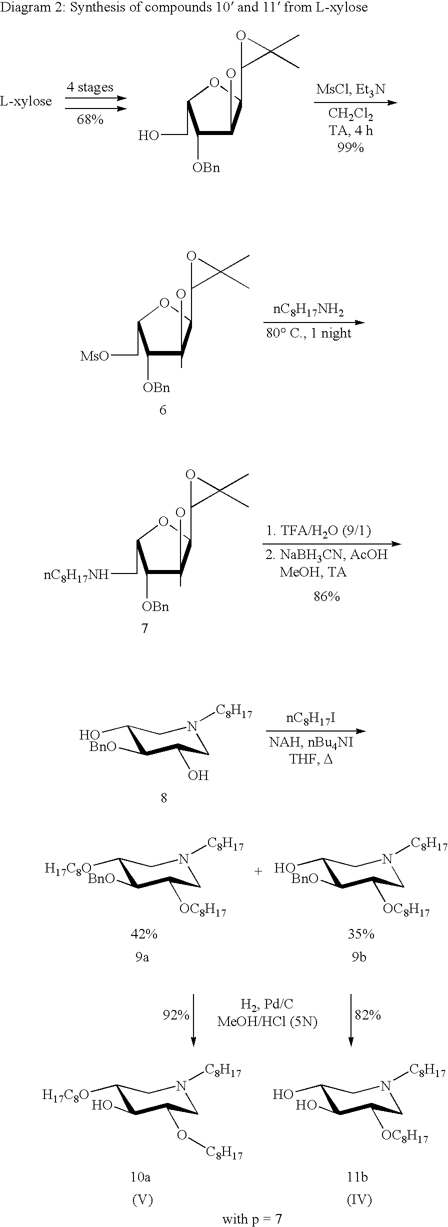

General Operating Method for Obtaining Mesylated Compound 6′

3-O-benzyl-1,2-O-isopropylidene-α-L-xylofuranose (1.78 g; 6.35 mmol) is solubilized in anhydrous dichloromethane (20 mL), at ambient temperature and under a current of argon. Then, triethylamine (1.1 mL; 7.89 mmol) and mesyl chloride (0.6 mL; 7.75 mmol) are added to the reaction medium. After stirring overnight, the organic phase is washed with water (1×20 mL) and a saturated sodium chloride solution (1×20 mL), then dried over magnesium sulphate, and concentrated under reduced pressure. The residue is chromatographed on silica gel with an elution gradient of petroleum ether/ethyl acetate (4/1→3/1→2/1) in order to produce the mesylated compound 6′ (2.26 g).

### Characteristics of Mesylated Compound 6′

Yield: 99%

Appearance: whitish solid

Rf: 0.2 (petroleum ether/ethyl acetate 4/1)

MS: m/z 359.5 [M+H]⁺ (theoretical 358.4)

HRMS (ESI): m/z 381.0983 [M+Na]⁺ (theoretical 381.0984)

[α]_{D}+45.5 (c=1.1, CHCl₃)

NMR ¹H (250 MHz, CDCl₃), δ(ppm):

1.32 (s, 3H); 1.48 (s, 3H); 2.99 (s, 3H); 4.00 (d, 1H, J=2.5 Hz); 4.44 (m, 4H); 4.66 (m, 2H); 5.95 (d, 1H, J=3.8 Hz); 7.28-7.39 (m, 5H)

NMR ¹³C (250 MHz, CDCl₃), δ(ppm):

26.3; 26.9; 37.5; 67.7; 72.0; 77.9; 81.5; 81.9; 105.3; 112.2; 127.9; 128.3; 128.7; 136.9

General Operating Method for Obtaining the Iminosugar 8′

Compound 6′ (1.56 g; 4.35 mmol) is solubilized in octylamine (10 mL) and the reaction medium is heated at 80° C. overnight, followed by coevaporation using toluene under reduced pressure in order to eliminate the excess of octylamine. The residue obtained is taken up in ethyl acetate (50 mL), the organic phase is washed with water (2×30 mL) and a saturated solution of sodium chloride (1×30 mL). This organic phase is then dried over sodium sulphate, and concentrated under reduced pressure. Octylamine still being present in the crude product obtained, aminated compound 7′ was therefore used in the following stages with no purification. Nevertheless, the mass and NMR ¹H spectra were produced have been realises on this intermediate crude amine:

MS: m/z 392.5 [M+H]⁺ (theoretical 391.6)

NMR ¹H (250 MHz, CDCl₃), δ(ppm):

0.88 (t, 3H, J=6.6 Hz); 1.15-1.50 (m, 18H); 2.60 (m, 2H); 2.90 (m, 2H); 3.90 (d, 1H, J=2.5 Hz); 4.31 (m, 1H); 4.48 (d, 1H, J=12.1 Hz); 4.62 (d, 1H, J=3.8 Hz); 4.70 (d, 1H, J=12.1 Hz); 5.93 (d, 1H, J=3.6 Hz); 7.32 (m, 5H)

Water is added to a solution of the crude amine 7′ obtained above in trifluoroacetic acid (60 mL) at 0° C., in order to obtain a solution 9/1 (v/v), under vigorous stirring. After 5 hours at ambient temperature, the reaction medium is coevaporated using toluene under reduced pressure, then the crude product is taken up in methanol (176 mL). Then acetic acid (0.52 mL; 9.1 mmol) and sodium cyanoborohydride (5.6 g; 89.1 mmol) are added to the reaction medium at 0° C. and under a current of argon. The solution is left to return to ambient temperature and stirred for 40 hours. Then, the solvent is evaporated under reduced pressure, the residue is taken up in dichloromethane (400 mL). The organic phase is washed with a saturated aqueous solution of sodium hydrogen carbonate (2×200 mL,), and with water (200 mL). Then it is dried over magnesium sulphate, and concentrated under reduced pressure. The residue obtained is chromatographed on silica gel with an elution gradient petroleum ether/ethyl acetate (1/1→1/2) in order to produce the expected iminosugar 8′ (1.25 g).

### Characteristics of the Iminosugar 8′

Yield: 86%

Appearance: white solid

Rf: 0.4 (petroleum ether/ethyl acetate 1/1)

MS: m/z 337.0 [M+H]⁺ (theoretical 335.5)

IR (v, cm⁻¹, NaCl): 3372; 2934; 2858; 1666; 1074; 1024

NMR ¹H (250 MHz, CDCl₃), δ(ppm):

0.88 (t, 3H, J=6.9 Hz); 1.26 (m, 10H); 1.46 (m, 2H); 2.20 (dd, 2H, J=8.5 and 11.0 Hz); 2.37 (m, 2H); 2.60 (m, 2H); 2.86 (dd, 2H, J=3.5 and 11.3 Hz); 3.25 (t, 1H, J=6.9 Hz); 3.77 (m, 2H); 4.78 (s, 2H); 7.26-7.37 (m, 5H)

NMR ¹³C (250 MHz, CDCl₃), δ(ppm):

14.2; 22.8; 26.9; 27.6; 29.4; 29.6; 31.9; 57.4; 58.0; 69.8; 74.0; 84.5; 127.9; 128.0; 128.7; 138.7

General Operating Method for Obtaining the Iminosugars 9′a and 9′b

The diol 8′ (1.25 g; 3.72 mmol) is solubilized in freshly distilled tetrahydrofuran (120 mL) at 0° C. and under a current of argon. Then 60% sodium hydride is added (0.76 g; 31.7 mmol) still at 0° C. The reaction medium is stirred for 30 minutes allowing the temperature to rise to ambient. Then iodooctane (5.4 mL; 29.7 mmol) and tetrabutylammonium iodide (0.28 g; 0.75 mmol) are introduced and the reaction is taken to reflux of the tetrahydrofuran for 28 hours. The excess of reagent is destroyed by slow addition of methanol, and the mixture is extracted with dichloromethane (2×50 mL). The organic phase is then washed with water (1×50 mL) and a saturated solution of sodium chloride (1×50 mL), dried over magnesium sulphate, and concentrated under reduced pressure. The residue is chromatographed on silica gel with an elution gradient petroleum ether/ethyl acetate (10/1→8/1→6/1→4/1→2/1) in order to produce the iminosugar 9′a (832.9 mg) and the racemic iminosugar 9′b (586.4 mg).

### Characteristics of the Iminosugar 9′a

Yield: 42%

Appearance: yellow oil

Rf: 0.7 (petroleum ether/ethyl acetate 10/1)

MS: m/z 561.0 [M+H]⁺ (theoretical 559.9)

IR (v, cm⁻¹, NaCl): 2930; 2864; 1674; 1272; 1099

NMR ¹H (250 MHz, CDCl₃), δ(ppm):

0.88 (m, 9H); 1.20-1.58 (m, 36H); 1.83 (t, 2H, J=10.7 Hz); 2.36 (m, 2H); 3.05 (dd, 2H, J=4.1 and 10.7 Hz); 3.21 (t, 1H, J=9.1 Hz); 3.38 (m, 2H); 3.59 (t, 4H, J=6.6 Hz); 4.83 (s, 2H); 7.21-7.40 (m, 5H)

NMR ¹³C (250 MHz, CDCl₃), δ(ppm):

14.2; 22.8; 26.3; 27.1; 27.6; 29.4; 29.6; 30.5; 31.9; 56.6; 58.2; 71.3; 75.3; 79.2; 86.4; 127.4; 127.9; 128.3; 139.5

### Characteristics of the Racemic Iminosugar 9′b

Yield: 35%

Appearance: yellowish solid

Rf: 0.15 (petroleum ether/ethyl acetate 10/1)

MS: m/z 449.0 [M+H]⁺ (theoretical 447.7)

IR (v, cm⁻¹, NaCl): 3418; 2930; 2855; 1638; 1376; 1100

NMR ¹H (250 MHz, CDCl₃), δ(ppm):

0.88 (m, 6H); 1.22-1.60 (m, 24H); 2.15 (m, 2H); 2.37 (m, 2H); 2.89 (m, 2H); 3.27 (t, 1H, J=7.2 Hz); 3.44-3.60 (m, 3H); 3.65 (m, 1H); 4.66 (d, 1H, J=11.6 Hz); 4.90 (d, 1H, J=11.6 Hz); 7.28-7.36 (m, 5H)

NMR ¹³C (250 MHz, CDCl₃), δ(ppm):

14.2; 22.8; 26.3; 27.0; 27.6; 29.5; 29.6; 30.3; 31.9; 55.4; 56.8; 58.2; 69.5; 70.4; 74.1; 78.5; 127.9; 128.7; 138.9

General Operating Method for Obtaining the Iminosugar 10′

The iminosugar 9′a (397.6 mg, 0.71 mmol) is solubilized, at ambient temperature and under a current of argon, in a mixture of methanol (20 mL) and hydrochloric acid 5N (2 mL). Then palladium on activated carbon is added to the reaction medium (10 mole %). The solution is then placed under vacuum, then under hydrogen. The mixture is stirred for 27 hours at ambient temperature, then filtered on a millipore filter, rinsed with methanol and concentrated under reduced pressure. The crude product is purified by silica gel chromatography with a petroleum ether/ethyl acetate mixture (10/1) in order to produce the desired iminosugar 10′ (306.5 mg).

### Characteristics of the Iminosugar 10′

Yield: 92%

Appearance: yellow oil

Rf: 0.2 (petroleum ether/ethyl acetate 10/1)

MS: m/z 471.0 [M+H]⁺ (theoretical 469.8)

HRMS (ESI): m/z 470.4585 [M+H]⁺ (theoretical 470.4573)

NMR ¹H (250MHz, CDCl₃), δ(ppm):

0.88 (m, 9H); 1.20-1.60 (m, 36H); 1.82 (t, 2H, J=10.0 Hz); 2.39 (m, 2H); 2.64 (m, 1H); 3.07 (dd, 2H, J=3.1 and 11.3 Hz); 3.29 (m, 3H); 3.57 (m, 4H)

NMR ¹³C (250 MHz, CDCl₃), δ(ppm):

14.2; 22.8; 26.2; 27.1; 27.6; 29.4; 29.5; 29.6; 30.3; 31.9; 55.6; 58.3; 70.7; 77.9; 78.6

General Operating Method for Obtaining the Racemic Iminosugar 11′

The racemic iminosugar 9′b (248.1 mg, 0.55 mmol) is solubilized, at ambient temperature and under a current of argon, in a mixture of methanol (15 mL) and hydrochloric acid 5N (1.5 mL). Then palladium on activated carbon is added to the reaction medium (10 mole %). The solution is then placed under vacuum, then under hydrogen. The mixture is stirred for 26 hours at ambient temperature, then filtered on a millipore filter, rinsed with methanol and concentrated under reduced pressure. The crude product is purified by silica gel chromatography with an ethyl acetate/methanol mixture (20/1) in order to produce the racemic iminosugar 11′ (161.2 mg).

### Characteristics of the Racemic Iminosugar 11′

Yield: 82%

Appearance: yellow oil

Rf: 0.5 (ethyl acetate/methanol 20/1)

MS: m/z 358.0 [M+H]⁺ (theoretical 357.6)

NMR ¹H (250 MHz, CD₃OD), δ(ppm):

0.93 (m, 6H); 1.22-1.45 (m, 20H); 1.46-1.62 (m, 4H); 1.86 (t, 1H, J=11.0 Hz); 1.90(t, 1H, J=11.0 Hz); 2.42 (m, 2H); 2.99 (dd, 1H, J=3.5 and 9.8 Hz); 3.11 (m, 1H); 3.16-3.31 (m, 2H); 3.51 (m, 1H); 3.62 (t, 2H, J=6.6 Hz)

NMR ¹³C (250 MHz, CD₃OD), δ(ppm):

14.5; 23.7; 27.2; 27.8; 28.6; 30.4; 30.5; 30.6; 31.2; 32.9; 33.0; 57.0; 59.1; 59.3; 71.4; 71.9; 79.4; 79.7

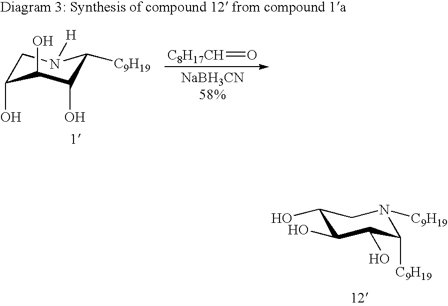

General Operating Method for Obtaining N-Alkylated Compound 12′

The iminosugar 1′a is solubilized (0.02 M), at ambient temperature and under a current of argon, in a methanol-acetic acid mixture (200/1, v/v). Then nonanal (1.2 eq) and sodium cyanoborohydride (1.2 eq) are added to the reaction medium followed by stirring overnight at ambient temperature. The solvents are then evaporated under reduced pressure. The residue obtained is taken up in ethyl acetate, the organic phase is washed with water, dried over magnesium sulphate and concentrated under reduced pressure. The crude product is purified by silica gel chromatography using the eluent ethyl acetate/methanol (5/1) and 1% triethylamine in order to produce the desired compound 12′. Yield: 58%

Characteristics of Compound 12′

Appearance: white solid

Rf: 0.35 (ethyl acetate/methanol 5/1+1% Et₃N)

[α]_{D}²⁰+5.5 (c 1.1, MeOH)

HRMS (ESI): m/z [M+H]⁺ calculated: 386.3634 found: 386.3633

HRMS (FAB): m/z [M+H]⁺ calculated: 386.3634 found: 386.3636

IR (NaCl, cm⁻¹): 1088 (C—O); 1150 (C—N); 2860-2928 (C—H); 3378 (O—H)

NMR ¹H (250 MHz, CD₃OD), δ(ppm):

0.91 (m, 6H); 1.31-1.51 (m, 30H); 2.46-2.68 (m, 3H); 2.75 (dd, 1H, J=4.7 and 12.6 Hz); 2.85 (m, 1H); 3.39 (t, 1H, J=8.5 Hz); 3.54 (m, 1H); 3.64 (dd, 1H, J=4.7 and 8.8 Hz)

NMR ¹³C (250 MHz, CD₃OD), δ(ppm):

14.5; 23.8; 24.3; 28.3; 28.5; 30.0; 30.4; 30.5; 30.6; 30.7; 31.0; 33.1; 52.5; 55.2; 63.0; 71.2; 72.6; 75.6

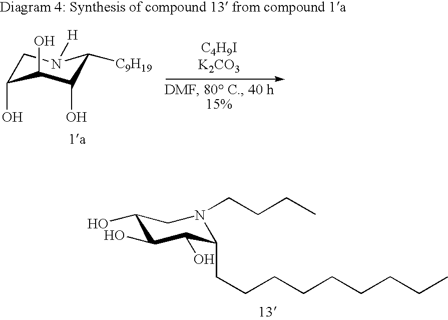

General Operating Method for Obtaining N-Alkylated Compound 13′

The crude iminosugar 1′a (26.5 mg; 0.102 mmol) is solubilized, at ambient temperature and under a current of argon, in anhydrous N,N-dimethylformamide (3.5 mL). Potassium carbonate (36 mg; 0.26 mmol) and 1-iodobutane (14 μL; 0.123 mmol) are added to the reaction medium. The latter is heated at 80° C. for 40 hours. The solvent is then co-evaporated using toluene and the residue obtained is chromatographed on silica gel with an ethyl acetate/methanol mixture (15/1) in order to produce the iminosugar 13′ (5 mg, 15%).

Characteristics of Compound 13′

Appearance: colourless oil

Rf: 0.35 (AcOEt/MeOH 15/1)

[α]_{D}²⁰+15.5 (c 0.4, MeOH)

HRMS (FAB): m/z [M+H]⁻ calculated: 316.2852 found: 316.2849

NMR ¹H 500 MHz (CD₃OD):

| δ (ppm) | Multiplicity | J (Hz) | Integration | Attribution |
|---|---|---|---|---|
| 0.90 | t | 6.6 | 3H | CH₃ alkyl |
| 0.93 | t | 6.6 | 3H | CH₃ alkyl |
| 1.25-1.57 | m | | 20H | 10 × CH₂ alkyl |
| 2.49 | dd | 4.2 and 12.5 | 1H | H-5 A |
| 2.54-2.66 | m | | 2H | CH₂N |
| 2.74 | dd | 4.1 and 12.9 | 1H | H-5 B |
| 2.84 | m | | 1H | H-1 |
| 3.39 | t | 7.8 | 1H | H-3 |
| 3.52 | m | | 1H | H-4 |
| 3.63 | dd | 4.4 and 8.2 | 1H | H-2 |

NMR ¹³C (CD₃OD)

δ (ppm) 14.4; 14.5 (2×CH₃ alkyl); 21.5; 23.8; 30.5; 30.7; 31.0; 33.1 (10×CH₂ alkyl); 49.3 (C-5); 52.5 (CH₂N); 55.1 (C-1); 63.2 (C-4); 71.3 (C-2); 72.8 (C-3)

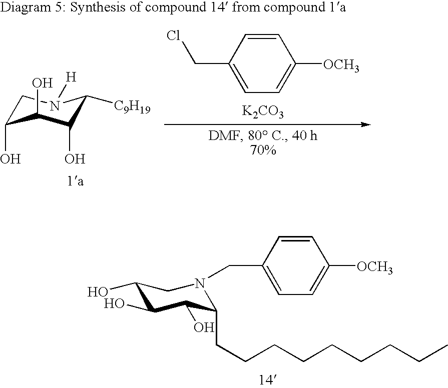

The crude iminosugar 1′a (28.3 mg; 0.11 mmol) is solubilized, at ambient temperature and under a current of argon, in anhydrous N,N-dimethylformamide (4 mL). Potassium carbonate (36.2 mg; 0.26 mmol) and p-methoxybenzyl chloride (18 μL; 0.13 mmol) are added to the reaction medium. The latter is heated to 80° C. overnight. The solvent is then co-evaporated using toluene and the residue obtained is chromatographed on silica gel with an ethyl acetate/methanol mixture (5/1) in order to produce the iminosugar 14′ (29 mg, 70%).

Characteristics of Compound 14′

Appearance: orange oil

Rf: 0.7 (AcOEt/MeOH 5/1)

[α]_{D}²⁰+17.0 (c 0.9, MeOH)

HRMS (FAB): m/z [M+H]⁺calculated: 380.2802 found: 380.2801

NMR ¹H (CD₃OD)

| δ (ppm) | Multiplicity | J (Hz) | Integration | Attribution |
|---|---|---|---|---|
| 0.92 | t | 6.9 | 3H | CH₃ nonyl |
| 1.30 | m | | 14H | 7 × CH₂ nonyl |
| 1.54 | m | | 2H | CH₂ nonyl |
| 2.50 | dd | 10.3 and | 1H | H-5 A |
| | | 12.3 | | |
| 2.68 | dd | 5.0 and 12.6 | 1H | H-5 B |
| 2.84 | m | | 1H | H-1 |
| 3.41 | t | 8.8 | 1H | H-3 |
| 3.56 | m | | 1H | H-4 |
| 3.71 | m | | 3H | H-2, CH₂N |
| 3.78 | s | | 3H | OCH₃ |
| 6.86 | d | 8.5 | 2H | H aromatic |
| 7.24 | d | 8.5 | 2H | H aromatic |

NMR ¹³C (CD₃OD)

δ (ppm) 14.5 (CH₃ nonyl); 23.7; 24.3; 29.8; 30.5; 30.7; 30.8; 30.9; 33.1 (8×CH₂ nonyl); 51.5 (C-5); 55.7 (OCH₃); 59.0 (CH₂N); 62.7 (C-1); 71.3 (C-4); 72.7 (C-2); 76.4 (C-3); 114.6; 130.9 (4×CH aromatic); 132.7; 160.3 (Cq aromatic)

II—Inhibition Tests on Human β-Glucocerebrosidase and Other Glycosidases

Inhibition tests on human δ-glucocerebrosidase were carried out in collaboration with Prof. N. Asano. The most relevant results are shown in Table 1. α-1-C-nonyl-XYL 1′a is currently the most powerful known inhibitor of human β-glucocerebrosidase with an inhibition constant (Kᵢ) of 2 nM. This compound is also extremely specific since it has no activity on the different α-glucosidases tested. This selectivity is due in part to the absence of a C-5 hydroxymethyl function which is characteristic of glucose. The extension of the length of the alkyl chain from C₉ to C₁₂ leads to a reduction in the inhibition activity. Similarly, the position of the nonyl group is crucial. N-nonyl iminoxylitol 6′ is thus 220 times less active than its C-alkylated analogue 1′a.

TABLE 1

| | IC₅₀ (μM) | | | |
|---|---|---|---|---|
| enzyme | 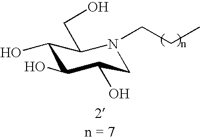 | 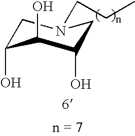 | 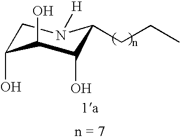 | 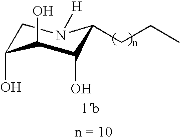 |
| α-glucosidase | | | | |
| rice | 0.08 | ND^{c} | NI | ND |
| yeast | NI^{b} | ND | NI | ND |
| maltase^{a} | 1.3 | ND | NI | ND |
| sucrase^{a} | 0.66 | ND | NI | ND |
| isomaltase^{a} | 0.23 | ND | NI | ND |
| β-glucosidase | | | | |
| β-glucocérébrosidase | 1.0 | 1.5 | 0.0068 (0.002)^{d} | 0.012 (0.031)^{d} |
| sweet almond | 150 | ND | 1.9 | ND |
| Caldocellum | 80 | ND | 800 | ND |
| saccharolyticum | | | | |

^{a}Rat intestine.
^{b}NI: less than 50% inhibition at 1000 μM.
^{c}ND Not determined.
^{d}value of Kᵢ.

Compound 6′ is a reference compound.

Compound 1′a corresponds to a compound of formula (I) in which R₀ represents a hydrogen atom, R₁ represents a nonyl group, R₂, R₃ and R₄ represent an OH group.

Compound 1′b corresponds to a compound of formula (I) in which R₀ represents a hydrogen atom, R₁ represents a dodecyl group, R₂, R₃ and R₄ represent an OH group.

The α-glucosidases of rice and of yeast, as well as the β-glucosidases of sweet almond and Caldocellum saccharolyticum are obtained from Sigma Chemical Co. ‘Brush border’ membranes prepared from rat small intestine according to Kessler's method (Kessler, M.; Acuto, O.; Strelli, C.; Murer, H.; Semenza, G. A. Biochem. Biophys. Acta 1978, 506, 136) were used as a source of intestinal maltase, sucrase and isomaltase. The activities of rice α-glucosidase as well as of the intestinal glucosidases were determined using an appropriate disaccharide as substrate. The D-glucose thus released was assayed by colorimetry using the Wako glucose B-test (Wako Pure Chemical Ind., Japan). The activities on the other glycosidases were determined using the appropriate p-nitrophenyl glycoside as a substrate with the optimum pH of each enzyme. The reaction is stopped by adding 400 mM Na₂CO₃. The p-nitrophenol thus released was assayed by spectrophotometry at 400 nm. For the β-glucocerebrosidase, the technique used is that described in Kato, A.; Kato, N.; Kano, E.; Adachi, I.; Ikeda, K.; Yu, L.; Okamoto, T.; Banba, Y.; Ouchi, H.; Takahata, H.; Asano, N. Biological properties of D- and L-1-deoxyazasugars. J. Med. Chem. 2005, 48, 2036-2044.

Biological Evaluation: Inhibition of Human β-glucocerebrosidase by the Compounds 12′, 13′ and 14′

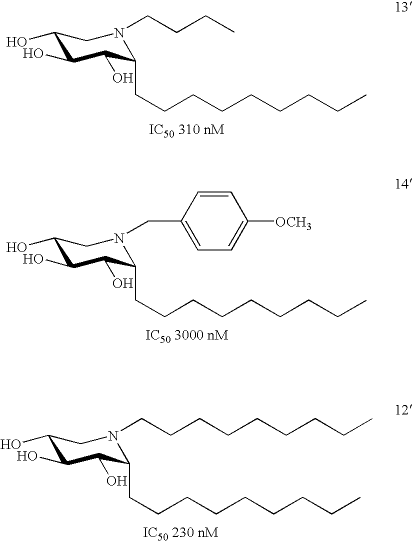

III—Effects of α-1-C-Nonyl-Iminoxylitol 1′a on the Intracellular Lysosomal Glycosidases in Fibroblasts of Patients Suffering from Gaucher's Disease (Type 1, 2 and 3)

A study was carried out to explore the effect of the inhibitors on the activity of intracellular p-glucocerebrosidases (fibroblasts originating from patients suffering from Gaucher's disease of Type 1, 2 or 3). Generally, an increase by a factor of 1.1 to 1.9 of the residual enzymatic activity was noted for the α-1-C-nonyl-XYL 1′a at a concentration comprised between 2.5 and 10 nM. (the main results are shown in Table 2).

In a remarkable manner, the use of α-1-C-nonyl-XYL (1′a) at a very low concentration of 10 nM made it possible to almost double the effectiveness of deficient β-glucocerebrosidases of type 1 and 3 (1.8 and 1.9 respectively) without inhibiting the action of other lysosomal glycosidases. It is to be noted that Gaucher's disease of type 1 is the most widespread. The comparative tests carried out with the N-nonyl DNJ 2′ showed that the increase in the enzymatic activity was also multiplied by a factor of 2 but at concentrations 1000 times higher (10 μM) and with poor selectivity vis-à-vis other glycosidases (α-Glucosidase and α-Mannosidase).

TABLE 2
Summary of the results obtained

| | Increase in the | Increase in the | Increase in the | |
|---|---|---|---|---|
| | enzymatic activity | enzymatic activity | enzymatic activity | Effects on the |
| For | (type 1 | (type 2 | (type 3 | activity of other |
| R = nC₉H₁₉ | fibroblasts) | fibroblasts) | fibroblasts) | glycosidases |
| 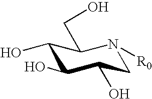 | ×2.4 to 10 μM | ×1.1 to 2.5 μM | ×1.6 to 10 μM | Marked effects |
| 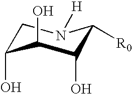 | ×1.8 to 10 nM | ×1.1 to 2.5 nM | ×1.9 to 10 nM | No effect |

Detailed Biological Tests:

The tests were carried out under the conditions described in: Sawkar, A. R.; Cheng, W.-C.; Beutler, E.; Wong, C.-H.; Balch, W. E.; Kelly, J. W. Chemical chaperones increase the cellular activity of N370S beta-glucosidase: a therapeutic strategy for Gaucher's disease. Proc. Natl. Acad. Sci. U.S.A. 2002, 99, 15428-15433.

Type 1 Gaucher Fibroblasts.

N-Nonyl-DNJ 2′
β-glucocerebrosidase: Residual activity increased by a factor of 2.4 to 10 μM.
α-Glucosidase: 70% inhibition at 50 μM.
α-Mannosidase: slight inhibition.

Type 2 Gaucher Fibroblasts.

N-Nonyl-DNJ 2′
β-glucocerebrosidase: Residual activity increased by a factor of 1.1 to 2.5 μM.
α-Glucosidase: significant inhibition at 50 μM.
α-Mannosidase: slight inhibition at 50 μM.

Type 3 Gaucher Fibroblasts

N-Nonyl-DNJ 2′
β-glucocerebrosidase: Residual activity increased by a factor of 1.6 to 10 μM.
α-Glucosidase: significant inhibition.
α-Mannosidase: no inhibition.

Type 1-3 Gaucher Fibroblasts

α-1-C-Nonyl-XYL 1′a
Type 1 β-glucocerebrosidase: Residual activity increased by a factor of 1.8 to 10 nM.
Type 2 β-glucocerebrosidase: Residual activity increased by a factor of 1.1 to 2.5 nM.
Type 3 β-glucocerebrosidase: Residual activity increased by a factor of 1.9 to 10 nM.
Type 1-3 α-Glucosidases: no inhibition.
Type 1-3 α-Mannosidase: no inhibition.

Conclusion

The results obtained with the α-1-C-nonyl-iminoxylitol 1′a open up the way towards future therapeutic agents which can be used against Gaucher's disease in a very small quantity and without side-effects. These compounds make it possible to significantly increase the residual enzymatic activity of β-glucocerebrosidase in Type 1 and 3 patients. It is also possible to envisage a bitherapy combining Zavesca®, in order to inhibit the formation of the glycosphingolipid involved, and a “chemical chaperone” activating the residual enzymatic hydrolysis activity of this glycolipid.

1. Method for the treatment of lysosomal diseases linked to a dysfunction of at least one lysosomal glycosidase enzyme comprising administering to a patient in need thereof of a compound of the following general formula (I): 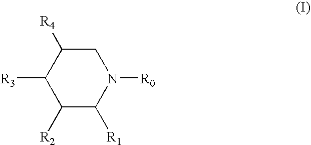
in which:
R₀ represents:
a hydrogen atom, or
a linear or branched, saturated or unsaturated alkyl group, comprising 1 to 12 carbon atoms, said alkyl group being optionally substituted by a phenyl group, if appropriate substituted by an alkoxy group comprising 1 to 15 carbon atoms, or
an oxaalkyl group comprising 3 to 12 members,
R₁ represents:
a hydrogen atom, or
a linear or branched, saturated or unsaturated alkyl group, comprising 4 to 16 carbon atoms, said alkyl group being optionally substituted by or carrying a substituent chosen from the following groups: hydroxyl, alkoxy comprising 1 to 12 carbon atoms and phenyl, or
an n-oxaalkyl group comprising 4 to 12 members, n representing an integer greater than or equal to 3,
R₂, R₃ and R₄ represent, independently of one another:
a hydrogen atom, or
a hydroxyl group, or
an alkoxy group of formula OR₅, R₅ representing a linear or branched, saturated or unsaturated alkyl group, comprising 1 to 15 carbon atoms or a benzyl group, or
an acyloxy group of formula O—CO—R₆, R₆ representing a linear or branched, saturated or unsaturated alkyl group, comprising 1 to 15 carbon atoms,
at least one of the groups R₁, R₂, R₃ and R₄ representing a linear or branched, saturated or unsaturated alkyl group, comprising 4 to 16 carbon atoms as defined above, or representing a group comprising a linear or branched, saturated or unsaturated alkyl group, comprising 1 to 15 carbon atoms as defined above,
said compound of formula (I) being in the form of a pure stereoisomer or in the form of a mixture of enantiomers and/or of diastereoisomers, including racemic mixture as well as their addition salts with pharmacologically acceptable acids,
providing that:
in formula (I), when R₀ represents a hydrogen atom or an alkyl group comprising 1 to 3 carbon atoms and R₁ represents a hydrogen atom, at least one of the groups R₂, R₃ and R₄ represents an alkoxy group, OR₅, or acyloxy group, OCOR₆, as defined above, in which R₅ or R₆ represents an alkyl group comprising at least 3 carbon atoms, and
in formula (I), at least two of the groups R₂, R₃ and R₄ do not represent a hydrogen atom.

2. Method according to claim 1 wherein R₀ represents a linear or branched, saturated or unsaturated alkyl group comprising 4 to 12 carbon atoms.

3. Method according to claim 2 wherein R₀ represents a linear or branched, saturated or unsaturated alkyl group comprising 6 to 12 carbon atoms.

4. Method according to claim 1 wherein R₅ represents a linear or branched, saturated or unsaturated alkyl group comprising 4 to 12 carbon atoms.

5. Method according to claim 1, wherein the lysosomal disease is the Gaucher's disease.

6. Method according to claim 1, wherein the lysosomal disease is the Krabbe's disease.

7. Method according to claim 1, wherein the lysosomal disease is the Fabry's disease.

8. Method according to claim 1, wherein R₀ represents a hydrogen atom.

9. Method according to claim 8, wherein R₁ represents an alkyl group comprising 4 to 16 carbon atoms.

10. Method according to claim 9 wherein R₁ represents an alkyl group comprising 9 carbon atoms.

11. Method according to claim 8 wherein R₂, R₃ and R₄ represent an OH group.

12. Method according to claim 9 wherein R₂, R₃ and R₄ represent an OH group.

13. Method according to claim 10 wherein R₂, R₃ and R₄ represent an OH group.

14. Method according to claim 1, wherein R₀ represents an alkyl group comprising 6 to 12 carbon atoms.

15. Method according to claim 14, wherein R₀ represents an alkyl group comprising 9 carbon atoms.

16. Method according to claim 14 wherein R₁ represents a hydrogen atom.

17. Method according to claim 14 wherein R₂ represents an alkoxy group of formula OR₅, R₅ representing an alkyl group comprising 3 to 15 carbon atoms.

18. Method according to claim 17 wherein R₂ represents an alkoxy group of formula OR₅, R₅ representing an alkyl group comprising 4 to 12 carbon atoms.

19. Method according to claim 16 wherein R₂ represents an alkoxy group of formula OR₅, R₅ representing an alkyl group comprising 3 to 15 carbon atoms.

20. Method according to claim 19 wherein R₂ represents an alkoxy group of formula OR₅, R₅ representing an alkyl group comprising 4 to 12 carbon atoms.

21. Method according to claim 14, wherein R₃ and R₄ represent OH groups.

22. Method according to claim 16, wherein R₃ and R₄ represent OH groups.

23. Method according to claim 17, wherein R₃ and R₄ represent OH groups.

24. Method according to claim 14 wherein R₃ represents an OH group and R₄ represents an alkoxy group of formula OR₅, R₅ representing an alkyl group comprising 3 to 15 carbon atoms.

25. Method according to claim 24 wherein R₃ represents an OH group and R₄ represents an alkoxy group of formula OR₅, R₅ representing an alkyl group comprising 4 to 12 carbon atoms.

26. Method according to claim 16 wherein R₃ represents an OH group and R₄ represents an alkoxy group of formula OR₅, R₅ representing an alkyl group comprising 3 to 15 carbon atoms.

27. Method according to claim 26 wherein R₃ represents an OH group and R₄ represents an alkoxy group of formula OR₅, R₅ representing an alkyl group comprising 4 to 12 carbon atoms.

28. Method according to claim 17 wherein R₃ represents an OH group and R₄ represents an alkoxy group of formula OR₅, R₅ representing an alkyl group comprising 3 to 15 carbon atoms.

29. Method according to claim 28 wherein R₃ represents an OH group and R₄ represents an alkoxy group of formula OR₅, R₅ representing an alkyl group comprising 4 to 12 carbon atoms.

30. Method according to claim 1 wherein the administered compound has the following general formula (II): 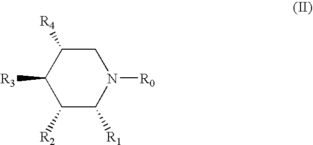
R₀, R₁, R₂, R₃ and R₄ being as previously defined.

31. Method according to claim 30, wherein the administered compound has the following formula (III): 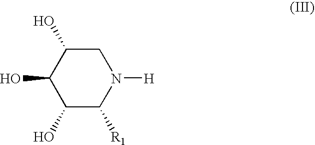
in which R₁ represents a linear or branched, saturated or unsaturated alkyl group, comprising 4 to 16 carbon atoms, said alkyl group being optionally substituted by or carrying a substituent chosen from the following groups: hydroxyl, alkoxy comprising 1 to 12 carbon atoms and phenyl

32. Method according to claim 31 wherein R₁ is a nonyl group.

33. Method according to claim 30, wherein the compound has the following formula (IV): 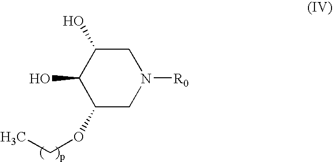
in which:
p represents an integer varying from 0 to 11,
R₀ represents a linear or branched, saturated or unsaturated alkyl group, comprising 1 to 12 carbon atoms, said alkyl group being optionally substituted by a phenyl group, if appropriate substituted by an alkoxy group comprising 1 to 15 carbon atoms.

34. Method according to claim 30, wherein the compound has the following formula (V): 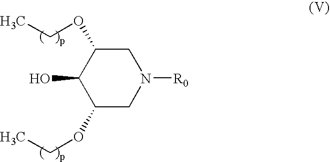
in which:
p represents an integer varying from 0 to 11,
R₀ represents a linear or branched, saturated or unsaturated alkyl group, comprising 1 to 12 carbon atoms, said alkyl group being optionally substituted by a phenyl group, if appropriate substituted by an alkoxy group comprising 1 to 15 carbon atoms.

35. Compound of the following general formula (I): 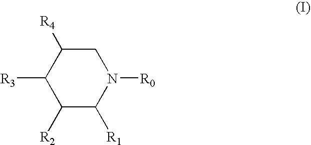
in which:
R₀ represents:
a hydrogen atom, or
a linear or branched, saturated or unsaturated alkyl group, comprising 1 to 12 carbon atoms said alkyl group being optionally substituted by a phenyl group, if appropriate substituted by an alkoxy group comprising 1 to 15 carbon atoms, or
an oxaalkyl group comprising 3 to 12 members,
R₁ represents:
a hydrogen atom, or
a linear or branched, saturated or unsaturated alkyl group, comprising 4 to 16 carbon atoms, said alkyl group being optionally substituted by or carrying a substituent chosen from the following groups: hydroxyl, alkoxy comprising 1 to 12 carbon atoms and phenyl, or
an n-oxaalkyl group comprising 4 to 12 members, n representing an integer greater than or equal to 3,
R₂, R₃ and R₄ represent, independently of one another:
a hydrogen atom, or
a hydroxyl group, or
an alkoxy group of formula OR₅, R₅ representing a linear or branched, saturated or unsaturated alkyl group, comprising 1 to 15 carbon atoms or a benzyl group, or
an acyloxy group of formula O—CO—R₆, R₆ representing a linear or branched, saturated or unsaturated alkyl group, comprising 1 to 15 carbon atoms,
at least one of the groups R₁, R₂, R₃ and R₄ representing a linear or branched, saturated or unsaturated alkyl group, comprising 4 to 16 carbon atoms as defined above, or representing a group comprising a linear or branched, saturated or unsaturated alkyl group, comprising 1 to 15 carbon atoms as defined above,
said compound of formula (I) being in the form of a pure stereoisomer or in the form of a mixture of enantiomers and/or of diastereoisomers, including a racemic mixture as well as their addition salts with pharmacologically acceptable acids,
providing that:
in formula (I), when R₀ represents a hydrogen atom or an alkyl group comprising 1 to 3 carbon atoms and R₁ represents a hydrogen atom, at least one of the groups R₂, R₃ and R₄ represents an alkoxy group OR₅ or acyloxy OCOR₆ as defined above, in which R₅ or R₆ represents an alkyl group comprising at least 3 carbon atoms, and
in formula (I), at least two of the groups R₂, R₃ and R₄ do not represent a hydrogen atom.

36. Method according to claim 35 wherein R₀ represents a linear or branched, saturated or unsaturated alkyl group comprising 4 to 12 carbon atoms.

37. Method according to claim 35 wherein R₀ represents a linear or branched, saturated or unsaturated alkyl group comprising 6 to 12 carbon atoms.

38. Method according to claim 35 wherein R₅ represents a linear or branched, saturated or unsaturated alkyl group comprising 4 to 12 carbon atom.

39. Compound according to claim 35, corresponding to the following formula (II): 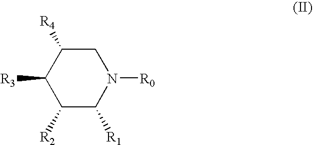
R₀, R₁, R₂, R₃ and R₄ being as previously defined.

40. Compound according to claim 35, corresponding to the following formula (III): 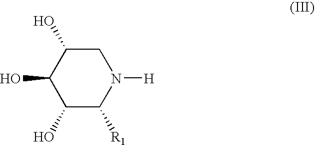
in which R₁ represents an alkyl group as previously defined.

41. Compound according to claim 35, corresponding to the following formula (IV): 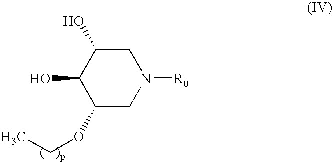
in which:
p represents an integer varying from 0 to 11,
R₀ represents an alkyl group as previously defined.

42. Compound according to claim 35, corresponding to the following formula (V): 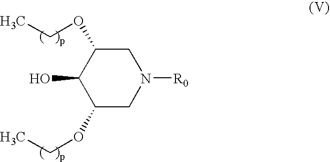
in which:
p represents an integer varying from 0 to 11,
R₀ represents an alkyl group as previously defined.

43. Composition pharmaceutical comprising a compound according to claim 35, in combination with a pharmaceutically acceptable vehicle.

44. Process for the preparation of a compound of formula (I) according to claim 35, in which R₁ represents an alkyl group or an n-oxaalkyl group, comprising the following stages:
a) the addition of an organometallic compound of formula R₁-M, in which R₁ represents an alkyl group or an n-oxaalkyl group as previously defined, M represents a metal or a group MgX in which X represents a halogen atom, on an imine of the following formula (1): 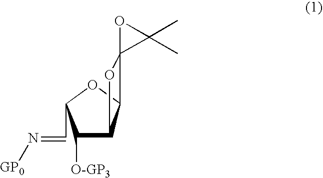
in which:
GP₀ represents a protective group selected from the group comprising the allyl, benzyl, p-methoxybenzyl and 2-naphthalenemethyl groups, and
GP₃ represents a protective group selected from the group comprising the allyl, benzyl and 2-naphthalenemethyl groups,
in order to obtain a compound of the following formula (2): 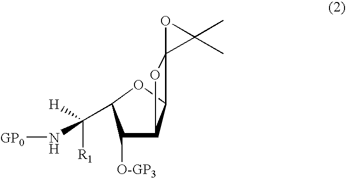
in which GP₀, GP₃ and R₁ are as defined above,
b) the hydrolysis in acid medium of the compound of formula (2) as defined above, followed by an intramolecular reductive amination reaction, in order to obtain a substituted piperidine of the following formula (3): 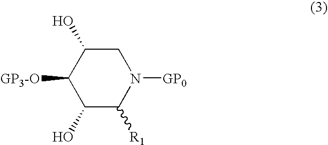
GP₀, GP₃ and R₁ being as defined above,
the compound of formula (3) being, if appropriate, deprotected in order to obtain a compound of formula (III),
said compound of formula (III) thus obtained being then optionally subjected to a stage of alkylation of the free amine function, said step being selected from the group comprising alkylation with an alkyl halide R₀X and reductive amination with an aldehyde originating from the oxidation of an alcohol of formula R₀OH, R₀ representing an alkyl group or an oxaalkyl group as previously defined, in order to obtain a compound of formula (II) in which R₂, R₃ and R₄ represent an OH group,
c) the protection of the free OH functions of the abovementioned compound (3), in order to obtain a substituted piperidine of the following formula (4): 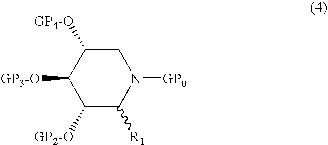
in which:
GP₂ represents a protective group selected from the group comprising the acetyl, benzoyl and pivaloyl groups,
GP₄ represents a protective group selected from the group comprising the trialkylsilyl groups, and
GP₀, GP₃ and R₁ are as defined above,
d) the chemoselective deprotection of one of the groups GP₂, GP₃ or GP₄, of the compounds of the abovementioned formula (4), in order to obtain respectively a compound of the following formula (5): 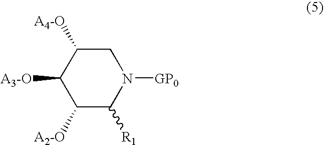
in which:
A₂ represents a hydrogen atom or a protective group GP₂,
A₃ represents a hydrogen atom or a protective group GP₃,
A₄ represents a hydrogen atom or a protective group GP₄,
and only one of the groups A₂, A₃ and A₄ represents H,
GP₀, GP₂, GP₃, GP₄, and R₁ being as defined above,
e) the reaction of the free hydroxyl function of the abovementioned compounds of formula (5),
either within the framework of the implementation of an alkylation process, selected from the group comprising alkylation with an alkyl halide of formula R₅—X, X representing a halogen atom, and R₅ representing an alkyl group comprising 3 to 15 carbon atoms, in order to obtain a compound of the following formula (6): 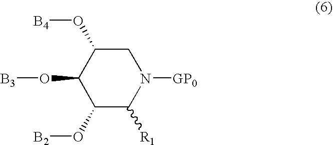
in which:
B₂ represents a group R₅ or a protective group GP₂,
B₃ represents a group R₅ or a protective group GP₃,
B₄ represents a group R₅ or a protective group GP₄,
and only one of the groups B₂, B₃ and B₄ represents R₅,
GP₀, GP₂, GP₃, GP₄, and R₁ being as defined above,
either within the framework of the implementation of an acylation process, for example with an acid chloride of formula R₆COCl, R₆ being as previously defined, in order to obtain a compound of the following formula (7): 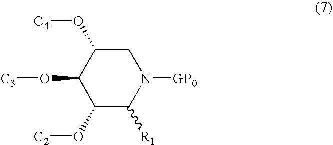
in which:
C₂ represents a group COR₆ or a protective group GP₂,
C₃ represents a group COR₆ or a protective group GP₃,
C₄ represents a group COR₆ or a protective group GP₄,
and only one of the groups C₂, C₃ and C₄ represents COR₆,
GP₀, GP₂, GP₃, GP₄, and R₁ being as defined above,
or within the framework of the implementation of a deoxidation process, for example by reaction with Im₂CS then Bu₃SnH, in order to obtain one of the compounds of the following formula (8): 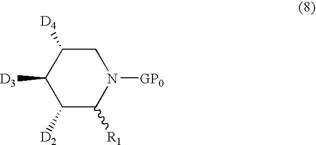
in which:
D₂ represents a hydrogen atom or a group OGP₂,
D₃ represents a hydrogen atom or a group OGP₃,
D₄ represents a hydrogen atom or a group OGP₄,
and only one of the groups D2, D₃ and D₄ represents H,
GP₀, GP₂, GP₃, GP₄, and R₁ being as defined above,
or within the framework of the implementation of a configuration inversion process, for example by the implementation of the Swern reaction, followed by reduction with a boron hydride, in order to obtain one of the compounds of the following formulae (9-1), (9-2) or (9-3): 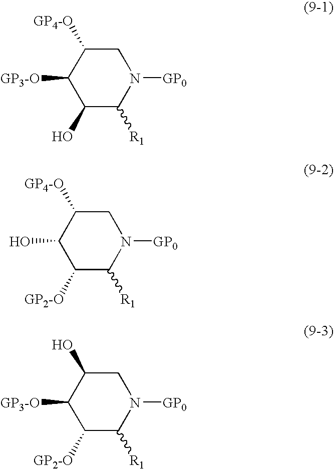
GP₀, GP₂, GP₃, GP₄, and R₁ being as defined above,
the abovementioned compounds of formulae (6), (7), (8), (9-1), (9-2) and (9-3) being able to be deprotected in order to obtain respectively the compounds of the following formulae (I-6), (I-7), (I-8), (I-9-1), (I-9-2) and (I-9-3), corresponding to compounds of formula (I) as previously defined: 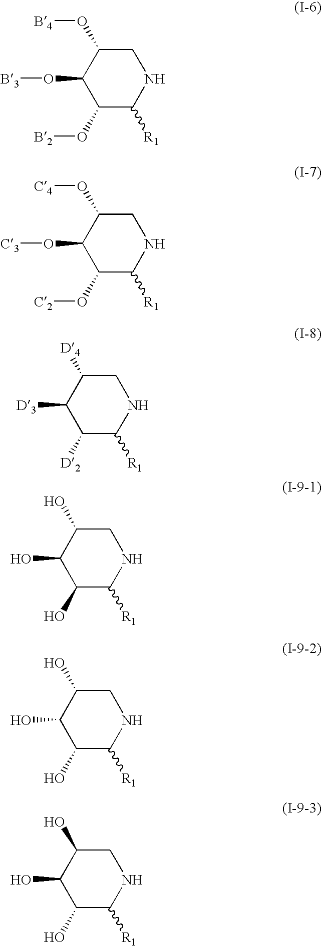
in which:
B′₂ represents a group R₅ or a hydrogen atom,
B′₃ represents a group R₅ or a hydrogen atom,
B′₄ represents a group R₅ or a hydrogen atom,
and only one of the groups B′₂, B′₃ and B′₄ represents R₅,
C′₂ represents a group COR₆ or a hydrogen atom,
C′₃ represents a group COR₆ or a hydrogen atom,
C′₄ represents a group COR₆ or a hydrogen atom,
and only one of the groups C′₂, C′₃ and C′₄ represents COR₆,
D′₂ represents a hydrogen atom or an OH group,
D′₃ represents a hydrogen atom or an OH group,
D′₄ represents a hydrogen atom or an OH group,
and only one of the groups D′₂, D′₃ and D′₄ represents H,
the compounds thus obtained of formulae (I-6), (I-7), (I-8), (I-9-1), (I-9-2) and (I-9-3) being able to then be optionally subjected to a stage of alkylation of the free amine function, for example by alkylation with an alkyl halide R₀X or by reductive amination with an aldehyde originating from the oxidation of an alcohol of formula R₀OH, R₀ representing an alkyl group or an oxaalkyl group as defined in claim 17, in order to obtain the compounds of the following formulae (I-6-a), (I-7-a), (I-8-a), (I-9-1-a), (I-9-2-a) and (I-9-3-a), corresponding to compounds of formula (I) as previously defined: 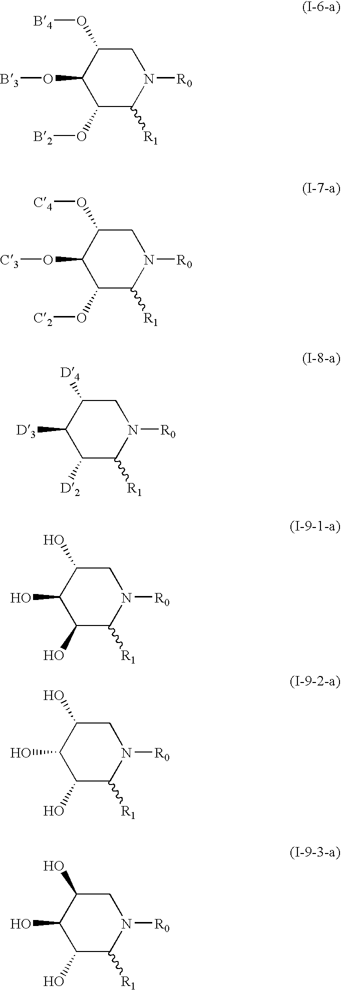
B′₂, B′₃, B′₄, C′₂, C′₃, C′₄, D′₂, D′₃, D′₄, R₀and R₁ being as defined above,
f) and, optionally, the regioselective deprotection of one of the remaining protective groups GP₂, GP₃ or GP₄, of the abovementioned compounds of formula (6), (7), (8), (9-1), (9-2) or (9-3) in order to obtain a deprotected free hydroxyl function, and the reaction of this free hydroxyl function, as described previously in Stage e), either within the framework of the implementation of an alkylation process, or within the framework of the implementation of an acylation process, or within the framework of the implementation of a deoxidation process, or within the framework of the implementation of a configuration inversion process, and an optional stage of deprotection in order to obtain compounds of formula (I) as defined above, optionally followed by a stage of alkylation of the free amine function, for example by alkylation with an alkyl halide R₀X or by reductive amination with an aldehyde originating from the oxidation of an alcohol of formula R₀OH, R₀ representing an alkyl group or an oxaalkyl group as previously defined, in order to obtain compounds of formula (I) in which R₀ is different from a hydrogen atom,
Stage f) being able to be optionally repeated if the compound obtained of formula (I) also contains a free hydroxyl function.

45. Process for the preparation of a compound of formula (I) according to claim 17, in which R₁ represents a hydrogen atom, comprising the following stages:
a) the reaction of a compound of the following formula (10): 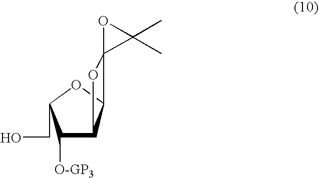
in which GP₃ represents a protective group in particular chosen from the allyl, benzyl and 2-naphthalenemethyl groups,
with a reagent containing an activating group Y, in order to obtain the compound of the following formula (11): 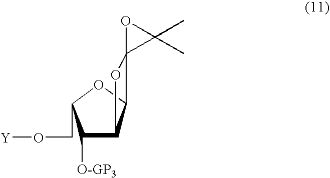
Y—O representing a leaving group, in which Y is an activating group in particular chosen from the mesyl, p-toluenesulphonyl and trifluoromethanesulphonyl groups,
b) the substitution of the compound of the abovementioned formula (11) by a primary amine R₀NH₂, R₀ representing an alkyl or oxaalkyl group as previously defined, in order to obtain a compound of the following formula (12): 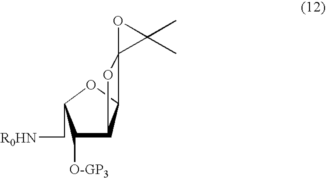
GP₃ being as defined above,
c) the hydrolysis in acid medium of the compound of formula (12) as defined above, followed by an intramolecular reductive amination reaction, in order to obtain a substituted piperidine of the following formula (13): 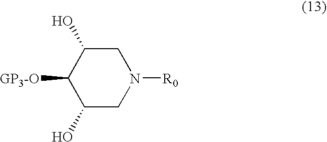
GP₃ and R₀ being as defined above,
d) the reaction of the compound of the abovementioned formula (13) with a compound of formula R₅X, R₅ being as previously defined and X representing a halogen atom, or with an acid chloride R₆COCl, R₆ being as previously defined, in order to obtain one of the following compounds (14-1) or (14-2): 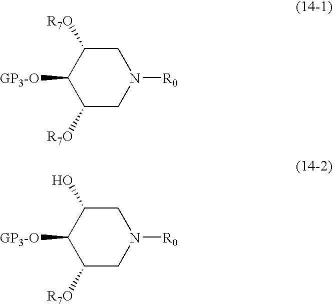
R₇ representing a group COR₆ or a group R₅ as defined above,
GP₃ and R₀ being as defined above,
the compounds of formulae (14-1) and (14-2) being in particular separated by silica gel chromatography,
the compound of formula (14-2) being, if appropriate, deprotected in order to obtain a compound of the following formula (I): 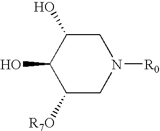
R₇ and R₀ being as defined above,
e) the regioselective deprotection of the group OGP₃ of the compound of formula (14-1) in order to obtain the compound of the following formula (14-3) containing a free hydroxyl function and corresponding to a compound of formula (I): 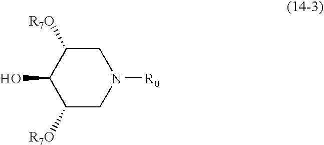
R₇ and R₀ being as defined above,
f) the reaction of the free hydroxyl function of the abovementioned compounds of formula (14-2) and (14-3),
either within the framework of the implementation of an alkylation process, for example with an alkyl halide of formula R₅—X, X representing a halogen atom, and R₅ representing a linear or branched, saturated or unsaturated alkyl group, comprising 1 to 15 carbon atoms, or a benzyl group, or
an acyloxy group of formula O—CO—R₆, R₆ representing a linear or branched, saturated or unsaturated alkyl group, comprising 1 to 15 carbon atoms,
in order to obtain respectively a compound of the following formula (15-2) or (15-3): 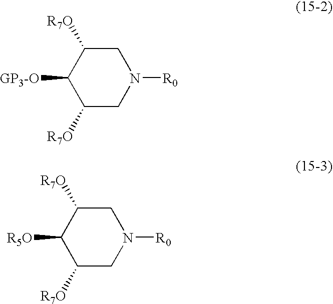
GP₃, R₅, R₇ and R₀ being as defined above,
the compound of formula (15-2) then being deprotected in order to obtain a compound of the following formula (I): 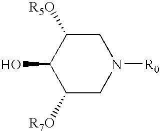
R₅, R₇ and R₀ being as defined above,
or within the framework of the implementation of an acylation process, for example with an acid chloride of formula R₆COCl, R₆ representing a linear or branched, saturated or unsaturated alkyl group, comprising 1 to 15 carbon atoms,
in order to obtain respectively a compound of the following formula (16-2) or (16-3): 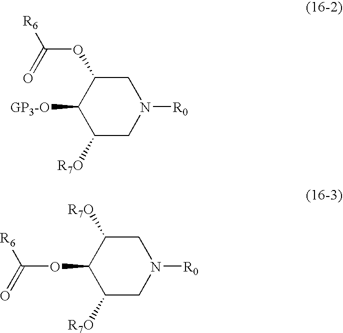
GP₃, R₆, R₇ and R₀ being as defined above,
the compound of formula (16-2) then being deprotected in order to obtain a compound of the following formula (I): 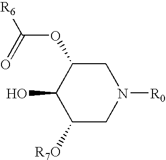
R₆, R₇ and R₀ being as defined above,
or within the framework of the implementation of a deoxidation process, for example by reaction with Im₂CS then Bu₃SnH, in order to obtain respectively a compound of the following formula (17-2) or (17-3): 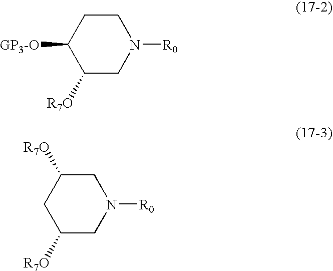
GP₃, R₇ and R₀ being as defined above,
the compound of formula (17-2) then being deprotected in order to obtain a compound of the following formula (I): 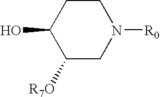
R₇ and R₀ being as defined above,
or within the framework of the implementation of a configuration inversion process, for example by the implementation of the Swern reaction, followed by reduction with a boron hydride, in order to obtain respectively a compound of the following formula (18-2) or (18-3): 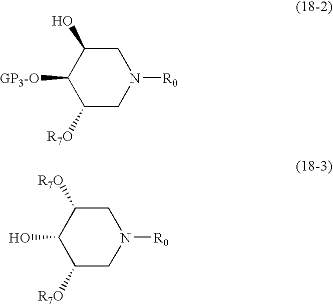
GP₃, R₇ and R₀ being as defined above,
the compound of formula (18-2) then being deprotected in order to obtain a compound of the following formula (I): 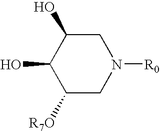
R₇ and R₀ being as defined above.
